SEQUENCE LISTING (38 sequences)

SEQ ID NO: 1 - DNA, 1098 bp, Homo sapiens
atgggggtcccgcggcctcagccctgggcgctggggctcctgctctttctccttcctgggagcctgggcgcagaaagccacctctccctcctgtaccaccttaccgcggtgtcctcgcctgccccggggactcctgccttctgggtgtccggctggctgggcccgcagcagtacctgagctacaatagcctgcggggcgaggcggagccctgtggagcttgggtctgggaaaaccaggtgtcctggtattgggagaaagagaccacagatctgaggatcaaggagaagctctttctggaagctttcaaagctttggggggaaaaggtccctacactctgcagggcctgctgggctgtgaactgggccctgacaacacctcggtgcccaccgccaagttcgccctgaacggcgaggagttcatgaatttcgacctcaagcagggcacctggggtggggactggcccgaggccctggctatcagtcagcggtggcagcagcaggacaaggcggccaacaaggagctcaccttcctgctattctcctgcccgcaccgcctgcgggagcacctggagaggggccgcggaaacctggagtggaaggagcccccctccatgcgcctgaaggcccgacccagcagccctggcttttccgtgcttacctgcagcgccttctccttctaccctccggagctgcaacttcggttcctgcggaatgggctggccgctggcaccggccagggtgacttcggccccaacagtgacggatccttccacgcctcgtcgtcactaacagtcaaaagtggcgatgagcaccactactgctgcattgtgcagcacgcggggctggcgcagcccctcagggtggagctggaatctccagccaagtcctccgtgctcgtggtgggaatcgtcatcggtgtcttgctactcacggcagcggctgtaggaggagctctgttgtggagaaggatgaggagtgggctgccagccccttggatctcccttcgtggagacgacaccggggtcctcctgcccaccccaggggaggcccaggatgctgatttgaaggatgtaaatgtgattccagccaccgcctga

SEQ ID NO: 2 - DNA, 1098 bp, Mus musculus
atggggatgccactgccctgggccctcagcctcttgttggtcctcctgcctcagacctggggctcagagacccgccccccactgatgtatcatctcacggctgtgtcaaacccatctacggggcttccctctttctgggctacaggctggttgggtcctcagcagtatctgacctacaacagcctgcggcaggaagctgacccctgtggggcctgggtgtgggaaaatcaggtgtcttggtattgggagaaggagaccacagacctcaaaagcaaagaacagctcttcttggaggccctcaagaccctggagaagatattaaatgggacctacacactgcagggcctgctgggctgtgaactggcctcggataattcctcagtgcccacggctgtgtttgccctcaatggtgaggagtttatgaaattcaacccaagaatcggcaattggactggggagtggcctgagacggaaatcgttgctaatctgtggatgaagcagcctgatgcggccaggaaggagagcgagttcctgctaaactcttgtccggagcgactgctaggccacctggagaggggccgacggaacctggagtggaaggagccgccgtctatgcgcctgaaggcccgtcctggcaactctggctcctccgtgctgacctgtgctgctttctccttctacccaccggagctcaagtttcgattcctgcgcaatgggctagcctcaggctccgggaattgcagcactggtccgaatggagatggctctttccacgcatggtcattgctggaggttaaacgtggagatgagcaccattatcaatgtcaagtggagcatgaggggctggcacagcctctcactgtggacctagattcatcagccagatcttctgtgcctgtggttggaatcgttcttggtttattgctggtggtagtggccatcgcaggcggtgtgctgttgtggggcaggatgcgcagcggtctgccagccccatggctttctctcagcggcgatgactctggtgacctgttgcctggtgggaacttgcccccagaagctgaacctcaaggtgcaaatgcctttccagccacttcctga

SEQ ID NO: 3 - DNA, 1101 bp, Rattus norvegicus
atggggatgtcccagcccggggtcctcctcagcctcttattggtcctcctgcctcagacctggggagcggagccccgtctcccactgatgtatcatcttgcagctgtgtctgacttatcaacggggcttccctctttctgggccacgggctggctgggtgctcagcaatatctgacctacaacaacctgcggcaggaggctgacccctgtggggcctggatatgggaaaaccaggtgtcttggtattgggagaaggagaccacggatctgaaaagcaaagaacagctcttcttggaggccatcaggaccctggagaaccaaataaatgggaccttcacactgcagggcctgctgggctgtgaactggcccctgataattcttcattgcccacggctgtgtttgccctcaatggtgaggagttcatgcggttcaacccaagaacgggcaactggagtggggagtggccggagacagatatcgttggtaatctgtggatgaagcaacctgaggcggccaggaaggagagcgagttcctgctaacttcttgtcctgagcggctgctaggccacctggagaggggccgtcagaacctggagtggaaggagccgccatctatgcgcctgaaggcccgtcctggcaactctggctcctcagtactgacctgtgctgctttctccttctacccgccggagctcaagtttcgattcctgcgcaatgggctagcctcaggctctgggaattgcagcactggtcccaatggtgatggatctttccatgcatggtcattgctagaggtcaaacgtggagatgaacaccattaccaatgtcaagtggagcatgaggggctggcccagcctctcactgtggacctagattcgcccgccagatcttctgtgcctgtggtcggaatcattcttggtttattgctggtggtagtggccatcgcagggggtgtgctgctatggaacaggatgcgaagtgggctgccagccccatggctttctctcagtggtgatgactctggcgacctattgcctggtgggaacttgcccccggaggctgaacctcaaggtgtaaatgcctttccggccacttcctga

SEQ ID NO: 4 - DNA, 1071 bp, Sus scrofa
atgcgggtcccccggcctcagccctggtggctcgctctgctgttcctcctgctgcccggaacgccgagcgcagacaaccaccgctccctcctgtaccacctcacggccgtgtccgcccccaccccgggcgctcccgccttctgggtgtcgggctggctggggccccagcagtacctgagctacaataacctgcgcgcccaggcggagccctacggcgcctgggtctgggaaagccaggtgtcctggtactgggagaaggagaccgcagacctgaggaacaagcagaagctctttctcgaggctctcaaaaccttggaggaaggagggcccttcaccctgcagggccttctgggctgcgaattgggccccgacaatgtctcggtgcctgtggccacgttcgccctgaatggcgaggagtttatgaagttcgacaccaagctgggcacctgggatggggagtggcccgaggcccggaccattggctccaagtggatgcaggagccggacgcggtcaacaaggagaagaccttcctgctctactcctgtccccaccggctgctggggcatctggagaggggccggggcaacctggagtggaaggagccgccctccatgcgcatgaaggcccgacccggcaccgcgccgggcttttccgtgctcacctgcatcgccttctccttctacccaccggagctgcagctgcgattcctccggaacgggctggcggctggctccggggagagtgacatcggccccaacggcgacggctccttccatgcctggtcgtcgctaacagtcaagagcggcgacgagcaccactactgctgtgtggtgcagcacgcggggctggctcagcccctcaccgtcgagctggaatccccagcgaagtcctccatgccagtggttggaatcatggtcggcttcctactgctcttgatcgtggctggaggaggagctctgctgtggaggaggatgaggaaggggctgccagccccttggatctcttttcatggggacgacgtaggggccctcctgcccacccccgacctggccaaggatgctgaatcttag

SEQ ID NO: 5 - DNA, 1065 bp, Bos taurus
atgcggcttccccggcctcagccctgggggctcggtctgctgctcgtcctcctgcctggggcgctgagcgcagagaactatcgctcgctccagtaccacttcaccgccgtgtcggcccccgcagcggggacccctgctttctgggtttcgggctggctggggccccagcagtacctgagctacaataatctacgggcgcaggctgagccctacggcgcgtgggtctgggaaagccaggtgtcctggtattgggagaaagagaccatggatctgaggaaccaggagacgctcttcctcgaagcgctccaagctttaggcgaaggccccttcaccatgcagggcctgttgggctgcgagttgggccctgacaatgtctcggtgccggtggccaagtttgccctgaacggcgaggagttcatgatgtttgaccccaagctgggcatctgggatggtgactggcctgagtcccggactgtcagtatcaagtggacgcagcagccagaggcggtcaacaaggagaagaccttcctcctctactcctgcccacaccggctgctggggcatctggagaggggccggggcaacctggagtggaaagagccaccctccatgcgcctgaaggcccgacccggcagtcccggcttctctgtgctcacctgcagcgccttctccttctacccacctgagctgaagctgcgctttctgcggaacgggctggccattggctctggtgagatagacatgggccccaacggcgacggctccttctacgcctggtcatcactcacagtcaagagtggcgacgagcaccactaccgctgcgtggtgcagcacgcggggctggcccagcccctcacggtggagctggaatcaccagccaggacctcggtgccagtggtggggatcgtcatcggcttattcctgctcctgacagtggctgcgggaggagctctcctgtggagaaggatgaggaaggggctgccagctccttggatctctttccgtggggaggatgtaggggccctcctgcccactcccggcctgtccaaggatggtgaatcttag

SEQ ID NO: 6 - DNA, 360 bp, Homo sapiens
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 7 - DNA, 360 bp, Mus musculus
atggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgtga

SEQ ID NO: 8 - DNA, 360 bp, Rattus norvegicus
atggctcgctcggtgaccgtgatctttctggtgcttgtctctctggccgtcgtgcttgccattcagaaaactccccaaattcaagtgtactctcgccatccaccggagaatgggaagcccaacttcctcaactgctacgtgtctcagttccacccacctcagatagaaattgagctactgaagaatggaaagaagataccaaatatcgagatgtcagatctgtccttcagcaaggactggtctttctacatcctggctcacactgaattcacacccaccgagaccgatgtatatgcttgcagagttaaacacgtcactctgaaggagcccaaaaccgtcacctgggaccgagacatgtaa

SEQ ID NO: 9 - DNA, 357 bp, Sus scrofa
atggctcccctcgtggccttggtcctgctcgggctgctctcactgtctggcctggatgcggtcgcgcgtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggatcagtatagctgccgcgtgaagcacgtgactctcgataagcccaagatagttaagtgggatcgagaccactaa

SEQ ID NO: 10 - DNA, 357 bp, Bos taurus
atggctcgcttcgtggccttggtccttctcgggctgctgtcgctgtctggactggacgccatccagcgtcctccaaagattcaagtgtactcaagacacccaccagaagatggaaagccaaattacctgaactgctatgtgtatgggttccatccaccccagattgaaattgatttgctgaagaatggggagaagattaaatcggagcagtcagacctgtctttcagcaaggactggtctttctacctgctgtcccacgctgagttcactcccaacagcaaggatcagtacagctgccgagtgaaacacgttactttggaacaaccccggatagttaagtgggatcgagacctgtaa

SEQ ID NO: 11 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 12 - protein, 365 aa, Mus musculus
MGMPLPWALSLLLVLLPQTWGSETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 13 - protein, 366 aa, Rattus norvegicus
MGMSQPGVLLSLLLVLLPQTWGAEPRLPLMYHLAAVSDLSTGLPSFWATGWLGAQQYLTYNNLRQEADPCGAWIWENQVSWYWEKETTDLKSKEQLFLEAIRTLENQINGTFTLQGLLGCELAPDNSSLPTAVFALNGEEFMRFNPRTGNWSGEWPETDIVGNLWMKQPEAARKESEFLLTSCPERLLGHLERGRQNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSPARSSVPVVGIILGLLLVVVAIAGGVLLWNRMRSGLPAPWLSLSGDDSGDLLPGGNLPPEAEPQGVNAFPATS

SEQ ID NO: 14 - protein, 356 aa, Sus scrofa
MRVPRPQPWWLALLFLLLPGTPSADNHRSLLYHLTAVSAPTPGAPAFWVSGWLGPQQYLSYNNLRAQAEPYGAWVWESQVSWYWEKETADLRNKQKLFLEALKTLEEGGPFTLQGLLGCELGPDNVSVPVATFALNGEEFMKFDTKLGTWDGEWPEARTIGSKWMQEPDAVNKEKTFLLYSCPHRLLGHLERGRGNLEWKEPPSMRMKARPGTAPGFSVLTCIAFSFYPPELQLRFLRNGLAAGSGESDIGPNGDGSFHAWSSLTVKSGDEHHYCCVVQHAGLAQPLTVELESPAKSSMPVVGIMVGFLLLLIVAGGGALLWRRMRKGLPAPWISFHGDDVGALLPTPDLAKDAES

SEQ ID NO: 15 - protein, 354 aa, Bos taurus
MRLPRPQPWGLGLLLVLLPGALSAENYRSLQYHFTAVSAPAAGTPAFWVSGWLGPQQYLSYNNLRAQAEPYGAWVWESQVSWYWEKETMDLRNQETLFLEALQALGEGPFTMQGLLGCELGPDNVSVPVAKFALNGEEFMMFDPKLGIWDGDWPESRTVSIKWTQQPEAVNKEKTFLLYSCPHRLLGHLERGRGNLEWKEPPSMRLKARPGSPGFSVLTCSAFSFYPPELKLRFLRNGLAIGSGEIDMGPNGDGSFYAWSSLTVKSGDEHHYRCVVQHAGLAQPLTVELESPARTSVPVVGIVIGLFLLLTVAAGGALLWRRMRKGLPAPWISFRGEDVGALLPTPGLSKDGES

SEQ ID NO: 16 - protein, 119 aa, Homo sapiens [sequence omitted]

SEQ ID NO: 17 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 18 - protein, 119 aa, Rattus norvegicus
MARSVTVIFLVLVSLAVVLAIQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 19 - protein, 118 aa, Sus scrofa
MAPLVALVLLGLLSLSGLDAVARPPKVQVYSRHPAENGKPNYLNCYVSGFHPPQIEIDLLKNGEKMNAEQSDLSFSKDWSFYLLVHTEFTPNAVDQYSCRVKHVTLDKPKIVKWDRDH

SEQ ID NO: 20 - protein, 118 aa, Bos taurus
MARFVALVLLGLLSLSGLDAIQRPPKIQVYSRHPPEDGKPNYLNCYVYGFHPPQIEIDLLKNGEKIKSEQSDLSFSKDWSFYLLSHAEFTPNSKDQYSCRVKHVTLEQPRIVKWDRDL

SEQ ID NO: 21 - protein, 330 aa, Homo sapiens [sequence omitted]

SEQ ID NO: 22 - protein, 5 aa, Artificial
  Polypeptide linker
GGGGS

SEQ ID NO: 23 - DNA, 44 bp, Artificial
  FCRN-KPNI-BSIWI-F
atcaggtacccgtacggccgccaccatgggggtcccgcggcctc

SEQ ID NO: 24 - DNA, 56 bp, Artificial
  FCRN-2xLINKER-BAMHI-R
agtcggatccgcctccgccgctgcctcctccgccggaggacttggctggagattcc

SEQ ID NO: 25 - DNA, 71 bp, Artificial
  FCRN-3xLINKER-BAMHI-R
agtcggatcctcctccgccgctgcctcctccgccgctgcctcctccgccggaggacttggctggagattcc

SEQ ID NO: 26 - DNA, 35 bp, Artificial
  B2M-BAMHI-F
agtcggatccatccagcgtactccaaagattcagg

SEQ ID NO: 27 - DNA, 50 bp, Artificial
  B2M-NOTI-MFEI-R
atcggcggccgccaattgttacatgtctcgatcccacttaactatcttgg

SEQ ID NO: 28 - DNA, 65 bp, Artificial
  B2M-BAMH1-2XLINKER-F
agtcggatccggcggaggaggcagcggcggaggcggctccatccagcgtactccaaagattcagg

SEQ ID NO: 29 - DNA, 45 bp, Artificial
  B2M-KPNI-BSIWI-F
caggtacccgtacggccgccaccatgtctcgctccgtggccttag

SEQ ID NO: 30 - DNA, 63 bp, Artificial
  B2M-2xLINKER-BAMHI-R
atcaggatccgcctccgccgctgcctcctccgcccatgtctcgatcccacttaactatcttgg

SEQ ID NO: 31 - DNA, 78 bp, Artificial
  B2M-3xLINKER-BAMHI-R
atcaggatccgcctccgccgctgcctcctccgccgctgcctcctccgcccatgtctcgatcccacttaactatcttgg

SEQ ID NO: 32 - DNA, 34 bp, Artificial
  FCRN-BCLI-F
acgtactgatcagcagaaagccacctctccctcc

SEQ ID NO: 33 - DNA, 43 bp, Artificial
  FCRN-NOTI-MFEI-R
atcggcggccgccaattgttaggaggacttggctggagattcc

SEQ ID NO: 34 - DNA, 64 bp, Artificial
  FCRN-BCL1-2XLINKER-F
atcagctgatcaggcggaggaggcagcggcggaggaggcagcgcagaaagccacctctccctcc

SEQ ID NO: 35 - DNA, 31 bp, Artificial
  FCRN-BCL1-F2
atggaatgatcattccacgcctcgtcgtcac

SEQ ID NO: 36 - DNA, 37 bp, Artificial
  B2M-XBAI-R
agcatctagagtaaacctgaatctttggagtacgctg

SEQ ID NO: 37 - DNA, 33 bp, Artificial
  B2M-XBA1-F
tcgttctagacatccagcagagaatggaaagtc

SEQ ID NO: 38 - DNA, 20 bp, Artificial
  FCRN-XBA1-R
ccctctagactcgagcggcc

(19) United States
(12) Patent Application Publication
(10) Pub. No.: US 2012/0021484 A1
(43) Pub. Date: Jan. 26, 2012

(54) Recombinant FcRn and Variants Thereof for Purification of Fc-Containing Fusion Proteins

(75) Inventor: Kevin A. McDonnell, Lexington, MA (US)

(73) Assignee: Biogen Idec MA Inc., Cambridge, MA (US)

(21) Appl. No.: 13/125,795

(22) PCT Filed: Oct. 21, 2009
(86) PCT No.: PCT/US2009/061517

§ 371 Date: Sep. 30, 2011

Related U.S. Application Data
(60) Provisional application No. 61/107,625, filed on Oct. 22, 2008.

(51) Int. Cl. C12N 9/00; C07K 1/22

(52) U.S. Cl. 435/183; 530/387.3; 530/383

(57) ABSTRACT

The invention is directed to methods of purifying Fc-containing molecules using a soluble neonatal Fc receptor (sFcRn). Native FcRn binds Fc-containing proteins at or below about pH 6.5 and releases them at or above about pH 7 and provides a much milder approach for capturing and purifying Fc-containing proteins, in particular, therapeutic Fc-containing proteins. Other embodiments of the invention provide modifications to alter the pH for binding and elution to the sFcRn, to modulate Fc-containing protein binding affinity, to affect sFcRn linkage to a support surface, or to improve the stability of sFcRn to conditions utilized in the methods of the invention.

### BACKGROUND OF THE INVENTION

1. Field of the Invention

The present invention relates to methods of purifying Fc-containing proteins using a soluble neonatal Fc receptor (sFcRn).

2. Background Art

Affinity chromatography is a powerful tool for the purification of proteins because of the ability of the affinity ligand to specifically bind a target molecule, e.g., an Fc-containing protein such as an antibody or Fc fusion protein. Antibodies and Fc fusion proteins both share an affinity for binding to Proteins A and G, which are often used as ligands in affinity purification because of their relatively high specificity for the binding partner. Use of Proteins A and G in the affinity purification of therapeutic antibodies or Fc fusion proteins can be problematic, however, because these ligands can leach into the eluted sample during purification. Protein A, for example, is immunogenic and potentially toxic in large amounts. In addition, some potentially therapeutic Fc fusion proteins an Fc moiety are not amenable to standard methods of purification because the conditions used to elute the purified protein are too harsh and cause structural or functional damage to the purified protein. For example, Protein A/G affinity chromatography, requires the use of strong acid (pH<4) or chaotropic agents to release the Fc-containing protein from the chromatographic media. The inventions described herein provide affinity purification methods using an alternative ligand, a soluble neonatal Fc receptor, that avoid the problems associated with the use of Proteins A and G.

Antibodies and other Fc fusion proteins bind to Fc receptors (FcR) through the Fc region. The Fc region of an IgG, for example, is comprised of paired C_{H2} and C_{H3} domains of IgG heavy chains, which form part of the larger IgG macromolecule. The overall structure of IgG may generally be characterized as a Y shaped molecule in which each upper arm of the Y is formed by a pairing of a single light chain (V_{L}C_{L}) with the two most amino terminal domains of a single heavy chain (V_{H}C_{H1}). The heavy and light chains are covalently bound to each other by a disulfide bond between the paired C_{L} and C_{H1} domains. The two heavy chains are covalently bound to each other through disulfide bonds in a region between the C_{H1} and C_{H2} domains known as the hinge region. The two heavy chains dimerize through interactions between their second and third constant domains (C_{H2} and C_{H3}) and bind the FcR.

Fc receptor is a general term that refers to any one of several proteins that bind to the Fc region of an immunoglobulin, one of which is the neonatal Fc receptor (FcRn). The FcRn is expressed on the luminal surface of intestinal epithelial cells. The physiological role of FcRn is to bind to maternal IgG consumed by the newborn when it drinks its mother's milk. The FcRn is then involved in the transport of the bound IgG across the intestinal epithelial barrier and the release of the IgG into the blood of the newborn. The FcRn was determined to optimally bind to IgG at the intestinal pH of 6-6.5 and to release bound IgG at the serosal pH of approximately 7.5.

FcRn is structurally similar to major histocompatibility complex (MHC) and consists of a heavy chain (α-chain) non-covalently bound to a light chain β2-microglobulin (β2m). The FcRn heavy chain has three extracellular domains (α1, α2, α3), a transmembrane domain, and a cytoplasmic tail. The three extracellular domains of the FcRn heavy chain have significant sequence similarity to the corresponding domains of Class I MHC molecules. The transmembrane domain of the FcRn heavy chain anchors the FcRn heterodimer into the cell membrane of the intestinal epithelial cells. The FcRn light chain is a soluble single domain protein also found as a component of the Class I MHC molecule heterodimer. The solved crystal structures of rat and human FcRn have confirmed the structural similarity of FcRn with Class I MHC molecules. See Burmeister et al. (2000) and West and Bjorkman (2000).

There are numerous potential applications for an FcRn which is soluble in aqueous solutions. Because the FcRn optimally binds to IgG at the intestinal pH of about 6-6.5 and releases bound IgG at the serosal pH of about 7.5, soluble FcRn can be used in IgG capture and release applications at physiological pHs. Such capture and release applications include affinity purification of therapeutic proteins, and in contrast to the harsh conditions required for Protein A/G affinity chromatography, provide a much milder approach for capturing and purifying these proteins. Thus, soluble FcRn of the kind disclosed and claimed herein can be attached to a surface and used to purify Fc-containing proteins from a sample to avoid the problems associated with the use of other modes of affinity chromatography.

### BRIEF SUMMARY OF THE INVENTION

The present invention provides a method of purifying Fc-containing proteins using a soluble neonatal Fc receptor (sFcRn) linked to a support surface. Because the FcRn binds Fc-containing proteins at or below about pH 6.5 and releases them at or above about pH 7, the use of FcRn in the purification of Fc-containing proteins provides a much milder approach for capturing and purifying these proteins. In particular, said method is of significant importance to the purification of therapeutic Fc-containing proteins, which were previously difficult to purify without causing structural or functional damage to the protein using the available purification methods, including Protein A/G chromatography.

In one embodiment, the present invention provides a method of purifying an enzymatically-active protein:Fc fusion protein using an sFcRn linked to a support surface. In a preferred embodiment, the present invention provides a method of purifying a Factor VIII:Fc fusion protein using an sFcRn linked to a support surface. Factor VIII:Fc is a fusion protein that comprises human clotting Factor VIII and Fc fragment from IgG1. Previous attempts to purify Factor VIII:Fc using standard approaches for purifying Fc fusion proteins have resulted in the complete loss of activity of the Factor VIII:Fc fusion protein. In accordance with the methods of the present invention, Factor VIII:Fc purified using an sFcRn linked to a support surface has increased purity and specific activity compared to prior methods of purification.

The present invention includes sFcRn immobilized by a number of chemical approaches to a variety of commercially available support surfaces. For example, the variety of surfaces includes but is not limited to, SEPHAROSE™, agarose, silica, collodion charcoal, sand, polystyrene, methacrylate, and other substrates capable of being linked or coupled to sFcRn (i.e., to form an “Fc binding phase”). Common chemical approaches for linking sFcRn to the surface include, for example without limitation, amide bond, disulfide bond formation, thioether bond formation, amine bond formation, ester bond formation, ether bond formation, urea bond formation, and thiourea bond formation. The sFcRn can also be covalently linked to a surface by carbon-carbon bond formation using radical based chemistry, including chemical, photo, or thermal activation.

The present invention further provides a method of purifying an Fc-containing protein using an sFcRn linked to a support surface wherein one or both heavy chain (α-chain) or light chain (β2m) domains are modified to modulate Fc-containing protein binding. For example, a higher affinity interaction could lead to a more efficient purification of dilute samples of Fc-containing proteins of interest. In a particular embodiment, the modification encompasses a mutation or mutations to one or both the α-chain or β2m of sFcRn, including mutations that either increase or decrease the binding affinity of Fc-containing proteins to sFcRn. In a preferred embodiment, the modification encompasses a mutation or mutations to one or both the α-chain or β2m of sFcRn that either increases or decreases the binding affinity of Factor VIII:Fc to sFcRn. In a particular embodiment, the modification encompasses a mutation or mutations to one or both the α-chain or β2m of sFcRn, including mutations that either increase or decrease the optimal pH for ligand binding and/or release. In a preferred embodiment, the modification encompasses a mutation or mutations to one or both the α-chain or β2m of sFcRn, including mutations that either increase or decrease the optimal pH for Factor VIII:Fc binding and/or release.

The present invention further provides a method of purifying an Fc-containing protein using an sFcRn linked to a support surface wherein the sFcRn α-chain or β2m are modified to modulate linkage to the surface. Such modification could allow for more efficient coupling to the chosen support. For example, a specific reactive group (e.g., cysteine) could be positioned in a location on the protein structure to result in a favorable attachment to the chosen support. This could result in a higher capacity for binding to the Fc protein or could result in a purification media more stable to multiple cycles of use. In a particular embodiment, the sFcRn linkage to a surface comprises an sFcRn modified with a specific reactive group. In another embodiment, the sFcRn linkage to a surface comprises a chemically, photo, or thermally activated cross-link. In a preferred embodiment, the modification to one or both the α-chain or β2m increases the purification efficiency of Factor VIII:Fc using sFcRn linked to a surface.

The present invention further provides a method of purifying an Fc-containing protein using an sFcRn linked to a support surface wherein the sFcRn α-chain or β2m are modified to improve the stability of the sFcRn to conditions required for multiple cycles of use. Covalently joining the two subunits to form a single chain sFcRn protein could result in greater stability towards the harsh conditions needed to sanitize a chromatographic media between cycles of use. For example, covalent cross-linking of the two subunits could be achieved by introducing cysteine residues at locations that would result in a disulfide bond between the α-chain and β2M chain domains or by using a chemically, photo, or thermally activated cross-linking reagent. In a particular embodiment, the sFcRn α-chain and β2m are covalently linked by a polypeptide or amino acid linker. In another embodiment, the sFcRn α-chain and β2m are chemically, photo, or thermally cross-linked. In a preferred embodiment, the modification to one or both the α-chain or β2m increases the purification efficiency of Factor VIII:Fc using the improved sFcRn.

The present invention further provides a method of purifying an Fc-containing protein using an sFcRn linked to a support surface wherein the method of purifying comprises a number of steps. Because the FcRn binds Fc-containing proteins at or below about pH 6.5 and releases them at or above about pH 7, a particular embodiment of the method comprises the steps of binding an Fc-containing protein to the sFcRn-linked surface at or below about pH 7.0 and the step of eluting said Fc-containing protein at or above about pH 7.0. Other alternative embodiments of the method are envisioned where binding of the Fc-containing proteins occurs at or above about pH 7.0 and eluting said Fc-containing proteins occurs at or below about pH 7.0. Such steps may be optimal where the sFcRn is modified to modulate Fc-containing protein binding affinity, to affect sFcRn linkage to the surface, or to improve the stability of sFcRn to conditions required for multiple cycles of use.

### BRIEF DESCRIPTION OF THE FIGURES

FIG. 1 shows an SDS-PAGE comparison of samples from a capture and release of Fc fragment using a soluble human FcRn (“shFcRn”)-SEPHAROSE™ column. Samples were run under non-reducing conditions on a 4-20% Tris-Glycine gradient gel. A solution containing pure Fc (Lanes 1, 19) was passed through the shFcRn-SEPHAROSE™ column. The unbound fraction was collected (Lane 2) and the column washed with pH 6 buffer (Lanes 3-8). The column was then eluted with pH 7.5 buffer, releasing the bound Fc (Lanes 9, 11-18). Molecular weight markers are in Lanes 10 and 20 (SEEBLUE® Plus2).

FIG. 2A shows the purification of Factor VIII:Fc using Protein A affinity chromatography. Samples were run under non-reducing conditions on an 8% Tris-Glycine gel. A crude sample containing Factor VIII:Fc was loaded onto a Protein A column (Load, Lane 2, 3). The unbound fraction was collected (Flow thru, Lanes 4-7). The column was washed (Lane 8-11), and then the bound Factor VIII:Fc was released by eluting the column with a gradient from pH 7.0 to pH 4.0 (Elution, Lanes 12-14) and from pH 4.0 to 3.0 (Lanes 15-20). The Factor VIII:Fc fusion protein is processed by cells into a light chain-Fc fusion (LC-Fc) and a heavy chain (HC) that are non-covalently associated via a metal coordination site. Molecular weight markers are in Lane 1 (SEEBLUE® Plus2).

FIG. 2B shows the Chromogenic Assay result for samples from purification of Factor VIII:Fc by Protein A affinity chromatography. Plot shows % recovery of Factor VIII clotting activity for the purification relative to the load (Lane 3). While the appropriate bands for Factor VIII:Fc are observed in FIG. 2A (Lanes 13-20), there is ˜1.6% total recovery of elution fractions (* fractions with Factor VIII:Fc present, Lanes 13-20).

FIG. 3A shows the purification of Factor VIII:Fc using shFcRn linked to NHS-activated SEPHAROSE™ 4 Fast Flow resin (“Fast Flow”). Samples were run under non-reducing conditions on a 4-12% Tris-Glycine gradient gel. A crude sample containing Factor VIII:Fc was adjusted to pH 6.0 and loaded onto the shFcRn-Fast Flow column (Load, Lane 1). The unbound fraction was collected (Flow thru, Lanes 2, 3). The column was washed with pH 6.0 buffer (Lane 4), and the bound Factor VIII:Fc was released by eluting the column with pH 7.5 buffer (Elution, Lanes 5-9). Molecular weight markers are in Lane 10 (SEEBLUE® Plus2).

FIG. 3B shows the Chromogenic Assay result for samples from purification of Factor VIII:Fc by shFcRn-Fast Flow chromatography. Plot shows % recovery of Factor VIII clotting activity for the purification relative to the load. Greater than 90% of the loaded activity was recovered in Elution fractions 1-5.

FIG. 4A shows a schematic representation of the single chain sFcRn constructs in either the light chain-linker-heavy chain orientation or the heavy chain-linker-light chain orientation.

FIG. 4B shows an SDS-PAGE comparison of samples collected from the expression and purification of the single chain sFcRn constructs. Lanes 1 and 2 contain wild-type sFcRn protein isolated from CHO cells and HEK 293-H cells, respectively. Lanes 3 through 6 contain the light chain-linker-heavy chain orientation constructs, with 2, 3, 4, and 5 GGGGS (SEQ ID NO: 22) linkers, respectively. Lanes 7 through 10 contain the heavy chain-linker-light chain orientation constructs, with 2, 3, 4, and 5 GGGGS (SEQ ID NO: 22) linkers, respectively.

FIG. 4C shows a Western Blot comparison of samples collected from the expression and purification of the single chain sFcRn constructs. Lanes 1 and 2 contain wild-type sFcRn protein isolated from CHO cells and HEK 293-H cells, respectively. Lanes 3 through 6 contain the light chain-linker-heavy chain orientation constructs, with 2, 3, 4, and 5 GGGGS (SEQ ID NO: 22) linkers, respectively. Lanes 7 through 10 contain the heavy chain-linker-light chain orientation constructs, with 2, 3, 4, and 5 GGGGS (SEQ ID NO: 22) linkers, respectively.

FIG. 5A shows an SDS-PAGE comparison of samples collected from the expression and purification of the single chain sFcRn variant constructs carrying a C48A or C251 A heavy chain mutation. Retained fractions from elution of the C48A-sFcRn (250 μg; 0.34 mg/mL) and C251A-sFcRn (51 μg; 0.34 mg/mL) variants were run on an SDS-PAGE gel. The transiently expressed C48A- and C251A-sFcRn variants eluted as single bands on SDS-PAGE gels (lanes marked “Elutions”). Lanes 1 and 2 contain samples of the supernatant and flow-through from the purification procedure (lanes marked “SUP” and “FT”).

FIG. 5B shows an SDS-PAGE comparison of samples collected from the expression and purification of the single chain sFcRn variant construct carrying an N102A heavy chain mutation. Retained fractions from elution of the N102A-sFcRn (6.65 μg; 0.19 mg/mL) variant were run under on an SDS-PAGE gel. The transiently expressed N102A-sFcRn variant eluted as a single band on an SDS-PAGE gel (lanes marked “Elutions”). Lanes 1 and 2 contain samples of the supernatant and flow-through from the purification procedure (lanes marked “SUP” and “FT”).

FIG. 5C shows a Western Blot comparison of samples collected from the expression and purification of the single chain sFcRn variant constructs carrying a C48A, C251A, or N102A heavy chain mutation. The purified N102A- (Lane 2), C48A- (Lane 3), and C251A- (Lane 4) sFcRn variants were visualized by immunoblotting with an anti-sFcRn antibody. Lane 1 contains a sample of single chain sFcRn.

FIG. 6A shows a schematic representation of the single chain sFcRn heterodimer dimer construct.

FIG. 6B shows an SDS-PAGE comparison of samples collected from the expression and purification of the single chain sFcRn construct in the light chain-linker-heavy chain orientation and the single chain sFcRn heterodimer dimer construct. Retained fractions from elution of the sFcRn heterodimer dimer construct (690 μg; 0.33 mg/mL) and single chain sFcRn light chain-linker-heavy chain construct (310 μg; 0.31 mg/mL) were run on an SDS-PAGE gel. The transiently expressed sFcRn heterodimer dimer construct (Lanes 11-15) and single chain sFcRn construct (Lanes 6-10) eluted as single bands on an SDS-PAGE gel. Lanes 2 through 5 contain samples of the supernatant and flow-through of the single chain sFcRn (Lanes 2 and 3) and sFcRn heterodimer dimer (Lanes 4 and 5) constructs from the purification procedure. Lane 1 contains molecular weight markers (SEEBLUE® Plus 2).

FIG. 7 shows a ribbon diagram of the 3-dimensional structure of FcRn with two serine residues (S⁵⁵ and S²⁷) at the heavy chain-light chain interface.

### DETAILED DESCRIPTION OF THE INVENTION

### Definitions

Terms are used herein as generally used in the art, unless otherwise defined as follows. In the case where there are two or more definitions of a term which is used and/or accepted within the art, the definition of the term as used herein is intended to include all such meanings unless explicitly stated to the contrary.

As used herein, the term “Fc-containing protein” is intended to include antibodies and Fc fusion proteins. Antibodies include whole antibody molecules, including monoclonal, polyclonal, and multispecific (e.g., bispecific) antibodies, as well as antibody fragments having the Fc region and retaining binding specificity and at least one effector function, and single chain antibodies, single chain Fv molecules, Fab fragments, diabodies, triabodies, tetrabodies, and the like. Also encompassed are chimeric and humanized antibodies, as well as antibodies engineered for use in other species. Fc fusion proteins, which can be recombinant or naturally occurring, include an Fc region or a region equivalent to the Fc region of an immunoglobulin and retain binding specificity and at least one effector function. An example of an Fc-containing protein is an enzymatically-active protein:Fc fusion protein, such as Factor VIII:Fc fusion protein. Enzymatically active proteins encompass polypeptides involved in a chemical reaction, including those polypeptides that catalyze the chemical reaction.

As used herein, the term “Fc region” is intended to refer to a C-terminal region of an IgG heavy chain. In a particular embodiment, the Fc region refers to the C-terminal region of a human IgG heavy chain (see, e.g., SEQ ID No.: 21). Although the boundaries of the Fc region of an IgG heavy chain might vary slightly, the human IgG heavy chain Fc region is usually defined to span from the amino acid residue at position Cys²²⁶ of the native polypeptide (or Cys¹⁰⁹ of SEQ ID No.: 21) to the carboxyl-terminus.

As used herein, the term “region equivalent to the Fc region of an immunoglobulin” is intended to include naturally occurring allelic variants of the Fc region of an immunoglobulin as well as genetically or artificially engineered variants having alterations which produce substitutions, additions, or deletions. For example, one or more amino acids can be deleted from the N-terminus or C-terminus of the Fc region of an immunoglobulin without substantial loss of biological function. Likewise, one or more amino acids can be inserted, deleted, or substituted within the Fc region without substantial loss of biological function. Such variants can be made according to biochemical principles known in the art so as to have minimal effect on activity.

As used herein, the terms “fusion” and “chimeric,” when used in reference to polypeptides such as an Fc fusion protein, refer to polypeptides comprising amino acid sequences derived from two or more heterologous polypeptides, such as portions of proteins encoded by separate genes (whether said genes occur in the same or a different species of organism).

As used herein, the term “variant” (or analog) refers to a polypeptide differing from a specifically recited polypeptide of the invention, such as FcRn, by amino acid insertions, deletions, and substitutions, created using, e.g., recombinant DNA techniques, such as mutagenesis. Guidance in determining which amino acid residues may be replaced, added, or deleted without abolishing activities of interest, may be found by comparing the sequence of the particular polypeptide with that of homologous peptides, e.g., human, primate, mouse, rat, bovine, porcine FcRn, and minimizing the number of amino acid sequence changes made in regions of high homology (conserved regions) or by replacing amino acids with consensus sequences.

Alternatively, recombinant polynucleotide variants encoding these same or similar polypeptides may be synthesized or selected by making use of the “redundancy” in the genetic code. Various codon substitutions, such as silent changes which produce various restriction sites, may be introduced to optimize cloning into a plasmid or viral vector for expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Amino acid “substitutions” may be the result of replacing one amino acid with another amino acid having similar structural and/or chemical properties, i.e., conservative amino acid replacements, or they may be the result of replacing one amino acid with an amino acid having different structural and/or chemical properties, i.e., non-conservative amino acid replacements. “Conservative” amino acid substitutions may be made on the basis of similarity in polarity, charge, solubility, hydrophobicity, hydrophilicity, or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions may be made by selecting the differences in polarity, charge, solubility, hydrophobicity, hydrophilicity, or the amphipathic nature of any of these amino acids. “Insertions” or “deletions” may be within the range of variation as structurally or functionally tolerated by the recombinant proteins. The variation allowed may be experimentally determined by systematically making insertions, deletions, or substitutions of amino acids in a polypeptide molecule using recombinant DNA techniques and assaying the resulting recombinant variants for activity.

As used herein, the term “effector function” refers to those biological activities attributable to the Fc region (a native sequence Fc region or amino acid sequence variant Fc region) of an Fc-containing protein. Examples of effector function include, but are not limited to, Fc receptor binding affinity; effector functions that operate after the binding of antibody to an antigen (these functions involve, for example, the participation of the complement cascade or FcR-bearing cells); and effector functions that operate independently of antigen binding (these functions confer, for example, persistence in the circulation and the ability to be transferred across cellular barriers by transcytosis).

As used herein, the term “host cell” covers any kind of cellular system which can be engineered to generate the polypeptides and antigen-binding molecules of the present invention, including sFcRn.

As used herein the term “native” (or naturally-occurring) polypeptide refers to an amino acid sequence that is identical to an amino acid sequence of an Fc region commonly found in nature. For example, native sequence human Fc regions include a native sequence human IgG1 Fc region (non-A and A allotypes); native sequence human IgG2 Fc region; native sequence human IgG3 Fc region; and native sequence human IgG4 Fc region, as well as, naturally occurring variants thereof. Other sequences are contemplated and are readily obtained from various databases (e.g., the National Center for Biotechnology Information (NCBI)).

The terms “Fc receptor” and “FcR” are used to describe a receptor that binds to an Fc region (e.g., the Fc region of an Fc-containing protein) or the functional equivalent of an Fc region. Portions of Fc receptors are specifically contemplated in some embodiments of the present invention. In one embodiment, the FcR is a native sequence human FcR. In other embodiments, the FcR is one which binds an IgG antibody (a gamma receptor) and includes receptors of the FcγRI, FcγRII, and FcγRIII subclasses, including allelic variants and alternatively spliced forms of these receptors. The term also includes the neonatal receptor, FcRn, which is responsible for the transfer of maternal IgGs to the fetus. A preferred embodiment of an Fc receptor encompassed by the present invention is the human neonatal Fc receptor (see SEQ ID Nos.: 1, 11 (α-chain) and 6, 16 (β2m)). Additional embodiments include, but are not limited to, FcRn from other species such as murine, rat, bovine, and porcine (see, e.g., SEQ ID Nos.: 2-5, 7-10 (α-chain) and 12-15, 17-20 (β2m)).

As used herein, unless indicated otherwise either explicitly or by context, the term “sFcRn” or “FcRn” is generally meant to indicate at least a portion of the extracellular region of the neonatal Fc receptor α-chain polypeptide and an associated β2m polypeptide (whether said polypeptides are covalently or non-covalently associated). “sFcRn” also includes native and variant α-chain and β2m polypeptides. Although the boundaries of the extracellular domain of FcRn α-chain may vary slightly, the human FcRn α-chain extracellular domain is usually defined to span from about the amino acid residues at positions Ala²⁴ to Ser²⁹⁷ (see SEQ ID NO: 11).

As used herein, an FcRn polypeptide variant with altered Fc binding affinity is one which has either enhanced (i.e., increased) or diminished (i.e., reduced) Fc binding affinity compared to a parent polypeptide or to a polypeptide comprising a native sequence FcRn. An FcRn polypeptide variant that exhibits increased binding affinity to an Fc binds at least one Fc with higher affinity than the parent polypeptide. A polypeptide variant that exhibits decreased binding affinity to an Fc, binds at least one Fc with lower affinity than a parent polypeptide.

The term “binding affinity” refers to the equilibrium dissociation constant (expressed in units of concentration) associated with each Fc receptor-Fc binding interaction. The binding affinity is directly related to the ratio of the kinetic off-rate (generally reported in units of inverse time, e.g., seconds⁻¹) divided by the kinetic on-rate (generally reported in units of concentration per unit time, e.g., molar/second). In general, changes in equilibrium dissociation constants due to differences in on-rates, off-rates, or both may be experimentally determined by techniques routinely used in the art (e.g., by BIACORE™ (www.biacore.com) or KINEXA® measurements (www.sapidyne.com)).

As used herein, the term “increased efficiency” or “increased purification efficiency,” when used in reference to modifications made to sFcRn, is intended to mean efficiency increases obtained with modifications to sFcRn compared to the efficiency of purification without the corresponding modifications. Thus, “increased purification efficiency” includes: for example, obtaining a more highly concentrated sample of Fc fusion proteins; obtaining a more highly concentrated sample of biologically active Fc fusion proteins (i.e., higher specific activity); obtaining a more highly purified sample of the Fc fusion protein (i.e., reduced levels of contaminants); and, increasing the stability of the FcRn-support surface, including without limitation, increased stability of the FcRn-support surface for re-use in multiple chromatographic purifications. Increased purification efficiency with respect to the above noted examples include increases of efficiency which may be in a range of about 1-10,000-fold greater than the efficiency obtained with unmodified FcRn; for example, efficiency increases may be 2-, 5-, 10-, 20-, 50-100-, 200-, 500-, 1000-, 2000-, 5000- or 10,000-fold. Increased purification efficiency also includes percent increases which may be in the range of about 20-100% greater than the efficiency obtained with unmodified FcRn; for example, percent increases may be 20%, 30%, 40%, 50%, 60%, 70%, 80%, 85%, 90%, 95%, 98%, and 99%.

As used herein, the term “support surface” includes any suitable surface or substrate that an FcRn may be coupled, or linked, to and provide for a means of purifying Fc-containing proteins, i.e., an Fc binding phase.

The term “biological activity,” when used in reference to the Fc-containing proteins of the invention, refers to the naturally or normally occurring functions associated with the Fc-containing proteins. An example of biological activity includes, but is not limited to, the specific activity of the Fc-containing proteins.

### Description of Methods

The present invention provides a method of purifying Fc-containing proteins using a soluble neonatal Fc receptor (sFcRn) linked to a support surface. Because the FcRn binds Fc-containing proteins at or below about pH 6.5 and releases them at or above about pH 7, the use of FcRn in the purification of Fc-containing proteins provides a much milder approach than other methods for capturing and purifying these proteins. In particular, said method is of significant importance to the purification of therapeutic Fc-containing proteins, which were previously difficult to purify without causing structural or functional damage to the protein using available purification methods, such as Protein A/G chromatography.

In one embodiment, the present invention provides a method of purifying Fc-containing proteins using affinity chromatography, where an adsorbent can comprise a suitable substrate with sFcRn affixed to its surface. A protein sample comprising the Fc-containing protein to be purified can be applied to this adsorbent. The adsorbent can be subsequently washed in a solution that does not interfere with binding of sFcRn to the Fc region of the Fc-containing protein. The Fc-containing protein can thereafter be eluted from the adsorbent with a solution that disrupts the binding of the Fc region to the sFcRn.

The present invention further provides a method of purifying an Fc-containing protein using sFcRn linked to a support surface wherein the purification method comprises a number of steps. Because FcRn binds Fc-containing proteins at or below about pH 6.5 and releases them at or above about pH 7, a particular embodiment of the method comprises the step of binding an Fc-containing protein to the sFcRn-linked surface at or below about pH 7.0 and the step of eluting said Fc-containing protein at or above about pH 7.0, respectively. Other alternative embodiments of the method are envisioned where FcRn variants are used which bind and elute Fc-containing proteins at pH levels other than about 7.0. For example, pH levels of at or below about 7.0 to about 4.0, such as at or below about 7.0, 6.9, 6.8, 6.7, 6.6, 6.5, 6.4, 6.3, 6.2, 6.1, 6.0, 5.9, 5.8, 5.7, 5.6, 5.5, 5.0, 4.5, or 4.0, may be used to bind Fc-containing proteins, and pH levels of at or above about 7.0 to about 10.0, such as at or above about 7.0, 7.1, 7.2, 7.3, 7.4, 7.5, 7.6, 7.7, 7.8, 7.9, 8.0, 8.1, 8.2, 8.3, 8.4, 8.5, 9.0, 9.5, or 10.0, may be used to elute Fc-containing proteins. Such steps may be optimal where sFcRn is modified to modulate Fc region binding affinity, to affect linkage to the support surface, or to improve stability of the sFcRn heterodimer when exposed to conditions required for multiple cycles of use.

Any or all chromatographic steps of the invention can be carried out by any mechanical means. Chromatography may be carried out in a column, for example. The column may be run with or without pressure and from top to bottom or bottom to top. The direction of the flow of fluid in the column may be reversed during the chromatography process. Chromatography may also be carried out using a batch process in which the support is separated from the liquid used to load, wash, and elute the sample by any suitable means, including gravity, centrifugation, or filtration.

The present invention includes the sFcRn immobilized to a variety of commercially available surfaces by a number of chemical approaches. The variety of commercially available surfaces includes, but is not limited to, SEPHAROSE™, agarose, silica, collodion charcoal, sand, polystyrene, methacrylate, and other substrates capable of forming an Fc binding phase. Common chemical approaches for linking sFcRn to the surface include, but are not limited to, amide bond formation, disulfide bond formation, thioether bond formation, amine bond formation, ester bond formation, ether bond formation, urea bond formation, thiourea bond formation. The sFcRn can also be covalently linked to the support by carbon-carbon bond formation using radical based chemistry, including chemical, photo, or thermal activation.

In a protein loading step, the sample (comprising the Fc-containing protein and contaminants) is loaded onto the adsorbent (comprising sFcRn affixed to a surface), in a solution comprising a buffer and/or a salt. Suitable buffers include, but are not limited to, phosphate buffers, amine buffers, acetate buffers, and citrate buffers. Suitable salts include, but are not limited to, sodium chloride, potassium chloride, ammonium chloride, sodium acetate, potassium acetate, ammonium acetate, calcium salts, and magnesium salts. For example, the solution may comprise MES at concentrations between about 5 mM and about 250 mM and sodium chloride at concentrations between about 50 mM and about 500 mM. However, other buffers and salts can be used. After protein loading, the adsorbent can be washed with more of the same solution. The protein can be eluted using a solution that disrupts binding of sFcRn to the Fc region of the Fc-containing protein. This “elution solution” may comprise a chaotropic agent, such as guanidinium, an agent that increases or decreases pH, or a salt. Elution may be effected by changing the pH of the solution. For example, the pH can be increased, to about 7.0 or above to elute Fc-containing proteins from naturally occurring sFcRn. Alternatively, a different change in pH (either up or down) may be required to elute Fc-containing proteins from sFcRn wherein one or both the α-chain and/or β2m are modified to improve purification efficiency. The elution solution may include any of the aforementioned buffers or salts. Solutions appropriate to effect elution may comprise, for example but without limitation, Tris at concentrations between about 5 mM and about 100 mM and sodium chloride at concentrations between about 50 mM and about 750 mM. Other methods of elution are also available, and conditions for binding and eluting can be readily optimized by those skilled in the art.

The Fc-containing protein, a complex of the protein and a second protein, or other proteins that may be present in a sample with the Fc-containing protein being purified, can be monitored by any appropriate means. For example, protein concentration of a sample at any stage of purification can be determined by any suitable method. Such methods are well known in the art and include without limitation: 1) colorimetric methods such as the Lowry assay, the Bradford assay, the Smith assay, and the colloidal gold assay; 2) methods utilizing the UV absorption properties of proteins; and 3) quantitation based on stained protein bands on gels relying on comparison with protein standards of known quantity on the same gel. Optionally, when sample purity is a critical factor, the technique should be sensitive enough to detect contaminants in the range between about 2 parts per million (ppm) (calculated as nanograms per milligram of the protein being purified) and 500 ppm. For example, enzyme-linked immunosorbent assay (ELISA), a method well known in the art, may be used to detect contamination of the Fc-containing protein by other proteins.

The Fc-containing protein can be produced by host cells that have been genetically engineered to produce the protein. Methods of genetically engineering cells to produce proteins are well known in the art. Such methods include introducing nucleic acids that encode and allow expression of the protein into living host cells. These host cells can be bacterial cells, fungal cells, insect cells, plant cells, or, preferably, animal cells grown in culture, to name only a few, and also include cells comprised within a transgenic animal, transgenic plant, or cultured plant or animal tissue. Bacterial host cells include, but are not limited to, Escherichia coli cells. Examples of suitable E. coli strains include without limitation: HB101, DH5α, GM2929, JM109, KW251, NM538, NM539, and any E. coli strain that fails to cleave foreign DNA. Fungal host cells that can be used include, but are not limited to, Saccharomyces cerevisiae, Pichia pastoris, and Aspergillus cells. A few examples of animal cell lines that can be used, but are not limited to, are CHO, VERO, BHK, HeLa, Cos, MDCK, HEK 293, 3T3, and WI138. New animal cell lines can be established using methods well know by those skilled in the art (e.g., by transformation, viral infection, and/or selection). Optionally, the protein can be secreted by the host cells into the medium.

The method of the invention may be used to purify Fc-containing proteins including antibodies or portions thereof and chimeric antibodies, e.g., antibodies having human constant antibody immunoglobulin domains coupled to one or more murine variable antibody immunoglobulin domain, or fragments thereof. Fc-containing proteins specifically contemplated for use with the invention include recombinant fusion proteins comprising one or more constant antibody immunoglobulin domains, preferably an Fc portion of an antibody, plus a protein, a receptor for any protein, or proteins substantially similar to such proteins or receptors. Other proteins that may be purified using the process of the invention include differentiation antigens (referred to as CD proteins) or their ligands or proteins substantially similar to either of these, which are fused to at least one constant antibody immunoglobulin domain, preferably an Fc portion of an antibody. Enzymatically active proteins (such as polypeptides involved in blood coagulation) or their ligands can also be purified according to the invention. Examples include, but are not limited to, recombinant fusion proteins comprising at least one constant antibody immunoglobulin domain plus all or part of one of the following proteins or their ligands or a protein substantially similar to one of these: metalloproteinase-disintegrin family members, various kinases, glucocerebrosidase, superoxide dismutase, tissue plasminogen activator, Factor VII, Factor VIII, Factor IX, Factor X, apolipoprotein E, apolipoprotein A-I, globins, an IL-2 antagonist, alpha-1 antitrypsin, TNF-alpha Converting Enzyme, Follicle-stimulating hormone, interferon beta, interferon alpha, ligands for any of the above-mentioned proteins, and numerous other proteins and their ligands.

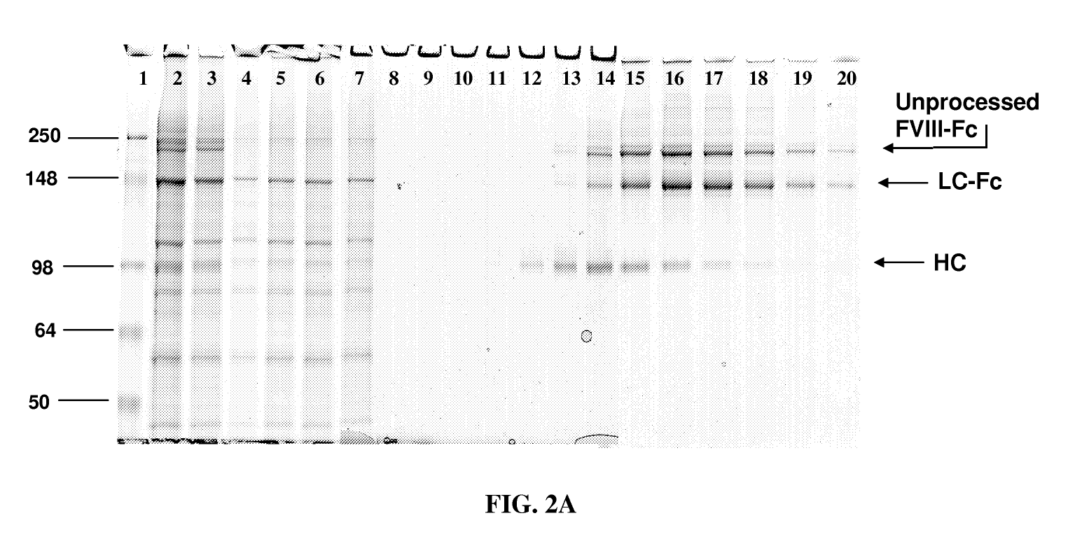

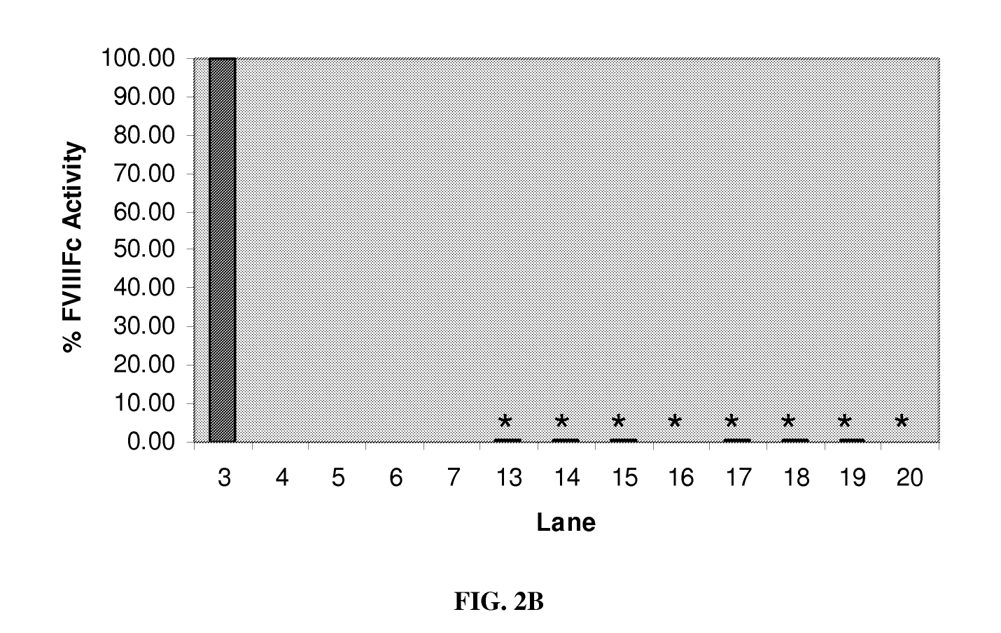

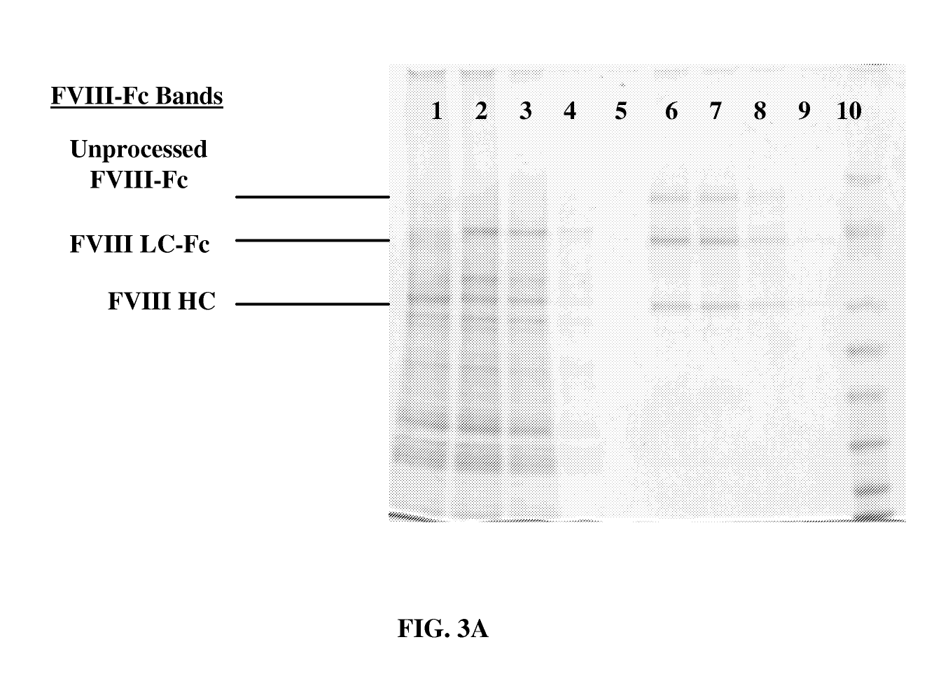

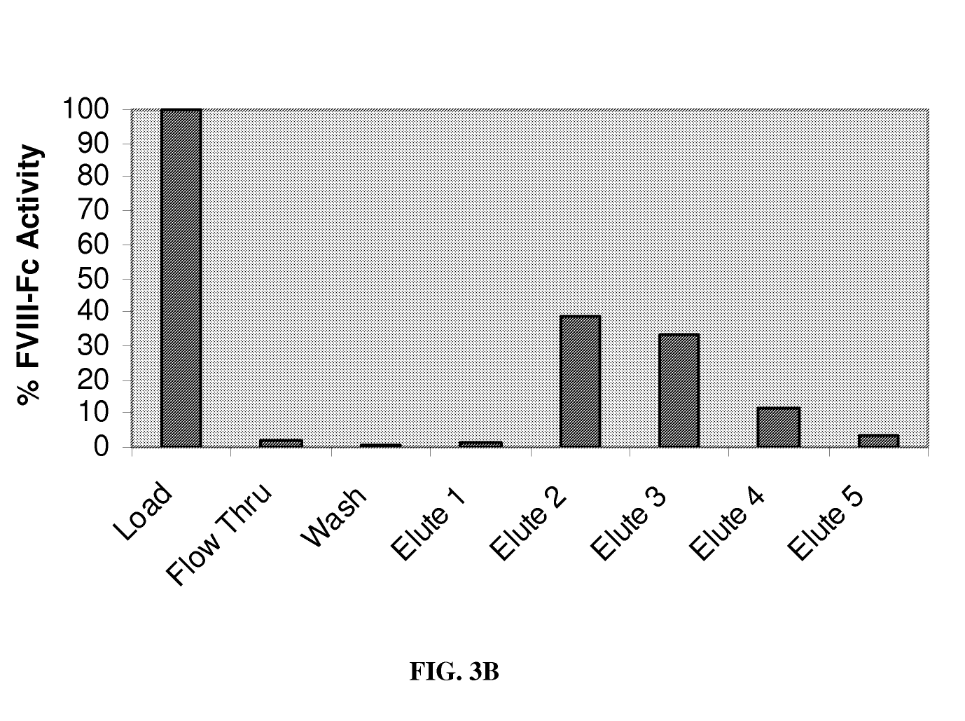

In a preferred embodiment, the present invention provides a method of purifying a Factor VIII:Fc fusion protein using an sFcRn linked to a support surface. Factor VIII:Fc is a fusion protein that comprises at least the bioactive portions of human clotting Factor VIII and the FcRn binding region of an immunoglobulin. Factor VIII is a glycoprotein cofactor synthesized and released into the bloodstream by the endothelium. In the circulating blood, it is mainly bound to von Willebrand factor to form a stable complex. Upon activation by thrombin (Factor IIa), it dissociates from the complex to interact with Factor IXa in the coagulation cascade. The lack of normal Factor VIII causes Hemophilia A. Previous attempts to purify Factor VIII:Fc using standard approaches for purifying Fc fusion proteins have resulted in the complete loss of activity of the Factor VIII:Fc protein. For example, purification of crude Factor VIII:Fc material using Protein A affinity chromatography, a standard approach for purifying Fc fusion proteins, results in a complete loss of activity even though the purity of the eluted Factor VIII:Fc is fairly high (see FIGS. 2A and 2B). In accordance with the methods of the present invention, Factor VIII:Fc purified using an sFcRn linked to a surface has increased purity and specific activity compared to prior methods of purification (see FIGS. 3A and 3B).

The present invention further provides a method of purifying an Fc fusion protein using an sFcRn linked to a support surface wherein one or both heavy chain (α-chain) or light chain (β2m) domains are modified to modulate Fc fusion protein binding. For example, a higher affinity interaction could lead to a more efficient purification of dilute samples of Fc-containing proteins of interest. In a particular embodiment, the modification encompasses a mutation or mutations to one or both the α-chain or β2m of sFcRn, including mutations that either increase or decrease the binding affinity of Fc-containing proteins to sFcRn. In a preferred embodiment, the modification encompasses a mutation or mutations to one or both the α-chain or β2m of sFcRn that either increases or decreases the binding of Factor VIII:Fc to sFcRn.

Modifications in Fc-containing protein binding affinity may be accomplished by selecting substitutions in FcRn that differ in their effect on maintaining (a) the structure of the FcRn polypeptide backbone in the area of the substitution (for example, as a sheet or helical conformation), (b) the charge or hydrophobicity of the molecule at the target site, or (c) the bulk of the side chain. Naturally occurring residues are divided into groups based on common side-chain properties: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Non-conservative substitutions will entail exchanging a member of one of these classes for another class. Such substituted residues also may be introduced into the conservative substitution sites or, more preferably, into the remaining (non-conserved) sites.

Substitutions to one or both the α-chain or β2m of sFcRn may be performed in accordance with standard techniques to provide variant nucleotide sequences. The substitutions can be made using methods known in the art such as oligonucleotide-mediated (site-directed) mutagenesis and random mutagenesis, such as scanning and PCR mutagenesis. Site-directed mutagenesis, cassette mutagenesis, restriction selection mutagenesis, or other known techniques can be performed on the cloned sFcRn DNA to produce the mutated α-chain or β2m.

Scanning amino acid mutagenesis can also be employed to identify one or more amino acids along a contiguous sequence for producing mutated α-chain or β2m that modulate Fc-containing binding affinity. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions. If alanine substitution does not yield adequate amounts of variant, an isosteric amino acid can be used.

The present invention further provides a method of purifying an Fc-containing protein using sFcRn linked to a support surface wherein the FcRn α-chain or β2m are modified to modulate linkage to the surface. Such modification could allow for more efficient coupling to the chosen support. For example, a specific reactive group (e.g., cysteine) could be positioned in a location on the protein structure of sFcRn to result in a favorable attachment to the resin. This could result in a higher capacity for binding to the Fc-containing protein or could result in a chromatographic media more stable to multiple cycles of use. In a particular embodiment, the sFcRn linkage to a surface comprises sFcRn modified with a specific reactive group. In another embodiment, sFcRn linkage to a surface comprises a chemically, photo, or thermally activated cross-link. In a preferred embodiment, the modification to one or both the α-chain or β2m increases the purification efficiency of Factor VIII:Fc using sFcRn linked to a surface.

Modifications of sFcRn for surface linkage include direct cross-linking of sFcRn to a surface. Cross-linking involves chemically joining two or more molecules by a covalent bond and is useful for solid-phase immobilization. Cross-linking reagents contain reactive ends to specific functional groups on proteins or other molecules. Cross-linking reagents include, but are not limited to, homobifunctional or heterobifunctional reagents. Homobifunctional cross-linking reagents have two identical reactive functional groups and often are used in one-step reaction procedures to cross-link proteins to each other or to stabilize quaternary structure. Heterobifunctional cross-linking reagents possess two different reactive groups that allow for sequential step-wise conjugations, which help to minimize undesirable polymerization or self-conjugation. Reactive functional groups of either class of reagents may be chemoreactive, photoreactive, or thermoreactive, without limitation.

Various types of commercially available cross-linkers are reactive with one or more of the following groups: primary and secondary amines (—NH), sulfhydryls (—SH), carbonyls (—C═O), carboxyls (—COOH), hydroxyls (—OH), silyls (e.g., bis(trimethoxysilyl) hexane), carbohydrates, or combinations thereof. All cross-linking reagents capable of binding FcRn to a surface are encompassed herein and include without limitation the following examples. Examples of amine-specific cross-linkers are bis(sulfosuccinimidyl) suberate, bis[2-(succinimidooxycarbonyloxy)ethyl]sulfone, disuccinimidyl suberate, disuccinimidyl tartarate, dimethyl adipimidate.2HCl, dimethyl pimelimidate.2HCl, dimethyl suberimidate.2HCl, and ethylene glycol bis(succinimidyl succinate). Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers reactive with carbonyl groups include 4-[4-azidosalicylamido]butylamine. Cross-linkers reactive with carbohydrates include azidobenzoyl hydrazine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include N-[k-maleimidoundecanoic acid]hydrazide, 4-(4-N-maleimidophenyl)butyric acid hydrazide HCl, and 3-[2-pyridyldithio]propionyl hydrazide.

Modifications of sFcRn for linkage to a support surface also include modifying the sFcRn polypeptides to effect favorable attachment to a surface. For example, the introduction of a specific reactive group (e.g., cysteine) on the protein structure of sFcRn can be accomplished by any methods known in the art, including mutagenesis as discussed herein. Such methods of modifying a reactive group on sFcRn polypeptides may also include post-translational chemical modification of amino acids in sFcRn, such as amine or thiol groups, so as to provide a point of attachment for a bifunctional cross-linker molecule. The modified sFcRn can then be cross-linked to the surface using any of the methods known in the art and as discussed herein, such as chemically, photo, or thermally cross-linking the modified sFcRn to the surface.

The present invention further provides a method of purifying an Fc fusion protein using an sFcRn linked to a support surface wherein the sFcRn α-chain or β2m are modified to improve the stability of the sFcRn to conditions required for multiple cycles of use. Covalently joining the two subunits to form a single chain sFcRn protein could result in greater stability towards the harsh conditions needed to sanitize a chromatographic media between cycles of use. For example, a covalent cross-linking of the two subunits could be achieved by introducing cysteine residues at locations that would result in a disulfide bond between the α-chain and β2M chain domains or by using a chemically, photo, or thermally active cross-linking reagent. In a particular embodiment, the FcRn α-chain and β2m are covalently linked by an amino acid linker. In another embodiment, the FcRn α-chain and β2m are chemically, photo, or thermally cross-linked. In a preferred embodiment, the modification to one or both the α-chain or β2m increases the purification efficiency of Factor VIII:Fc using the improved sFcRn.

Modification of the α-chain or β2m to improve the stability of the sFcRn to conditions required for multiple cycles of use include any methods known in the art or as discussed herein. For example, the α-chain or β2m could be modified to promote disulfide bond formation between the α-chain and β2m by introducing cysteine residues using methods such as site-directed mutagenesis. The FcRn α-chain or β2m can be fused directly using chemically, photo, or thermally reactive cross-linking reagents to join the α-chain and β2m. Alternatively, the α-chain or β2m can be linked by any suitable linker, such as a polypeptide linker. Alternatively, an amino acid linker sequence may be employed to link the polypeptides. Such amino acid linker sequences can be incorporated into the α-chain and β2m using standard techniques well known in the art. Suitable peptide linker sequences may be chosen based on the following factors, including but not limited to: (1) their ability to adopt a flexible extended conformation; (2) their ability or inability to adopt a desired secondary or tertiary structure; and (3) the presence or absence of hydrophobic, charged and/or polar residues. Non-limiting examples of amino acids that can be used in peptide linker sequences include glycine, valine, serine, alanine, or threonine residues. In various embodiments, a linker sequence may generally be from about 1, 2, 3, 4, 5, 6, 7, 8, 9, 10, 15, 20, 25, 30, 35, 40, 45 to about 50 amino acids in length but can be about 100 to about 200 amino acids in length or higher.

### EXAMPLES

### Example 1

### Affinity Purification of Fc-Containing Proteins Using a Soluble Neonatal Fc Receptor (sFcRn) Column

The soluble neonatal Fc Receptor (sFcRn) column can be used for the purification of Fc-containing proteins, and Fc fusion proteins from crude or partially purified media extracts. The sFcRn column is prepared by incubating sFcRn and a commercially available substrate via any number of chemical approaches. For example, covalent coupling to a support surface can be through formation of amide bonds, disulfide bonds, thioether bonds, amine bonds, ester bonds, ether bonds, urea bonds, or thiourea bonds. The sFcRn protein can also be covalently linked to a surface by carbon-carbon bond formation using chemically, photo, or thermally activated chemistry.

The resulting sFcRn-linked substrate is washed with a buffered solution at or below pH about 7.0 and poured as an affinity purification column. The sFcRn column is then equilibrated with a buffer solution at or below pH about 7.0.

Crude or partially-purified media extracts comprising Fc-containing proteins are buffered using a buffered solution at or below pH about 7.0, and the resulting solution is applied to the sFcRn column. Following application of the buffered media extracts to the column, the column is washed with 5-10 column volumes of a buffered solution at or below pH about 7.0. Affinity bound Fc-containing proteins are then eluted with a buffer solution at or above pH about 7.0 and collected in fractions of appropriate volume.

### Example 2

### Capture and Release Analysis of a Purified Fc-Containing Proteins Using Soluble Human Neonatal Fc Receptor (shFcRn)

An shFcRn column can be tested for Fc binding affinity by applying a purified Fc molecule in a buffer solution at pH 6.0. After washing the column, the affinity bound Fc molecule is eluted with a buffer solution at pH 7.5.

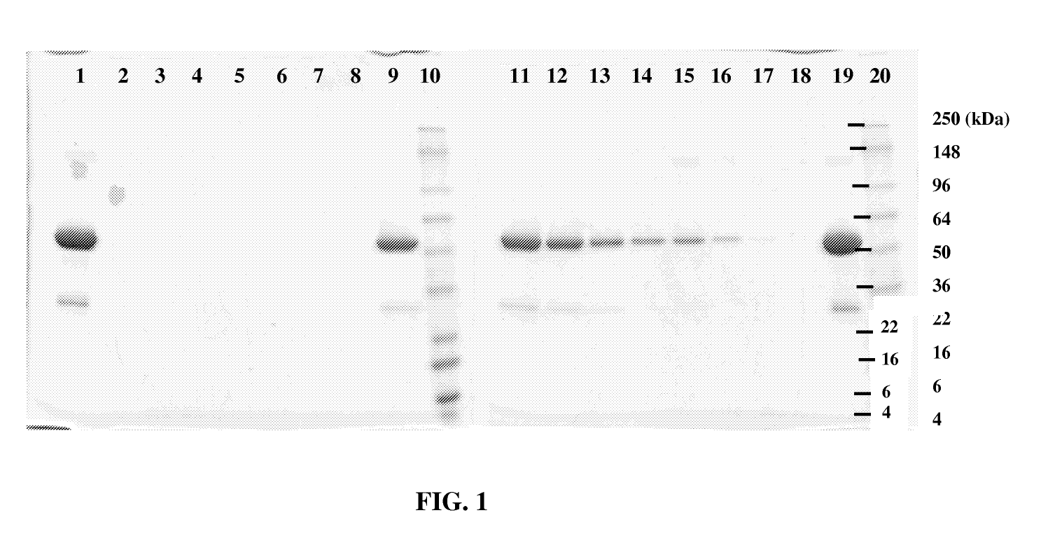

Samples from the capture and release of a purified Fc molecule were analyzed by SDS-polyacrylamide gel electrophoresis (SDS-PAGE), as illustrated in FIG. 1. Retained fractions from elution of the purified Fc molecule from an shFcRn-SEPHAROSE™ resin column were run under non-reducing conditions on a 4-20% Tris-Glycine gradient gel. The bound Fc molecule eluted with the pH 7.5 buffer (Lanes 9, 11-18) as expected, instead of with the pH 6 buffer in the unbound column flow-thru (Lane 2) and column washes (Lanes 3-8). Lanes 1 and 19 contain a sample of pure Fc molecule, and Lanes 10 and 20 contain molecular weight markers (SEEBLUE® Plus 2, Invitrogen). These results illustrate the ability of an shFcRn column to bind a purified Fc molecule at pH 6 and then to elute it at pH 7.5.

### Example 3

### Purification of Factor VIII:Fc Fusion Protein Using Soluble Human Neonatal Fc Receptor (shFcRn)

An shFcRn column was used for the purification of the Fc fusion protein Factor VIII:Fc, which comprises human clotting factor VIII and an Fc fragment from IgG1. shFcRn (5 mg/mL) was covalently coupled to NHS-activated SEPHAROSE™ 4 Fast Flow resin (“Fast Flow”; GE Healthcare) via amide bond formation by incubating shFcRn and Fast Flow at pH 7.5 for 1-2 hours at room temperature. The resulting shFcRn-Fast Flow (10 mg shFcRn/mL Fast Flow) was washed and poured as an affinity purification column. The column (10 cm bed height) was then equilibrated with a buffer solution of 50 mM MES, 100 mM NaCl, and 2 mM CaCl₂ at pH 6.0 (“Buffer Solution A”).

Crude or partially-purified Factor VIII:Fc fusion protein was buffered to 167 mM MES, pH 6.0, using a solution of 1 M MES at pH 6.0, and the resulting solution was applied to the column at a flow rate of 50-60 cm/hour. Following application of Factor VIII:Fc fusion protein to the column, the column was washed with 5-10 column volumes of Buffer Solution A at a flow rate of 50-60 cm/hour. Affinity bound Factor VIII:Fc was then eluted with a buffer solution of 25 mM Tris, 500 mM NaCl, and 2 mM CaCl₂ at pH 7.5.

Analysis of Factor VIII:Fc Fusion Protein Purified Using Soluble Human Neonatal Fc Receptor (shFcRn)

Samples from the affinity purification of the Factor VIII:Fc fusion protein were analyzed by SDS-PAGE, as illustrated in FIG. 3A. Retained fractions from elution of the Factor VIII:Fc fusion protein were run under non-reducing conditions on a 4-12% Tris-Glycine gradient gel. The bound Factor VIII:Fc fusion protein eluted with the pH 7.5 buffer (Lanes 6-9) instead of in the unbound column flow-thru (Lanes 2-3) or column wash with pH 6 buffer (Lane 4). Lane 1 contains a sample of crude or partially-purified Factor VIII:Fc fusion protein in pH 6 buffer, and Lane 10 contains molecular weight markers (SEEBLUE® Plus 2). These results illustrate the ability of the shFcRn-Fast Flow column to significantly purify the Factor VIII:Fc fusion protein from a crude or partially-purified sample as demonstrated by the enrichment of the unprocessed Factor VIII:Fc band in Lanes 6-8.

Samples from the affinity purification of the Factor VIII:Fc fusion protein were analyzed in a Chromogenic Assay, as illustrated in FIG. 3B. Retained fractions from elution of the Factor VIII:Fc fusion protein were analyzed for Factor VIII activity. As compared to the unbound column flow-thru and column wash with pH 6 buffer, greater than 90% of the loaded activity was recovered in Elution fractions 1-5, with the pH 7.5 buffer.

The shFcRn-Fast Flow column purified Factor VIII:Fc fusion protein has an observed peak specific activity of ˜9,000 IU/mg (Table 1). In comparison, a recombinant Factor VIII product available on the market (REFACTO®) has a reported specific activity of 12,000 IU/mg. Adjusting for the added mass of the Fc fusion protein to Factor VIII, the shFcRn-Fast Flow column purified Factor VIII:Fc fusion protein exhibits almost 100% of the activity of REFACTO®, 1980 IU/nmol compared to 2040 IU/nmol, respectively (Table 1). These results illustrate the ability of the shFcRn-Fast Flow column, and associated purification conditions, to significantly purify a highly active Factor VIII:Fc fusion protein.

TABLE 1
Specific activity of Factor VIII:Fc fusion protein purified by an
shFcRn-Fast Flow column compared to recombinant Factor
VIII (REFACTO ®)

| | IU/mg | MW (kDa) | IU/nmol |
|---|---|---|---|
| Factor VIII:Fc | 9,000 | 220,000 | 1,980 |
| REFACTO ® | 12,000 | 170,000 | 2,040 |

### Example 3

### Cloning and Expression of Single Chain Soluble Neonatal Fc Receptor (sFcRn) Proteins

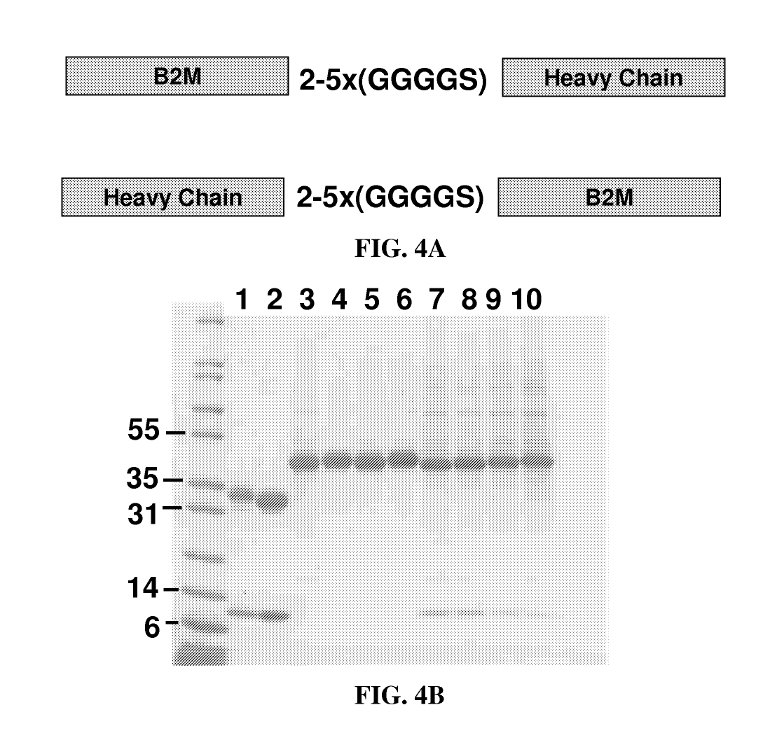

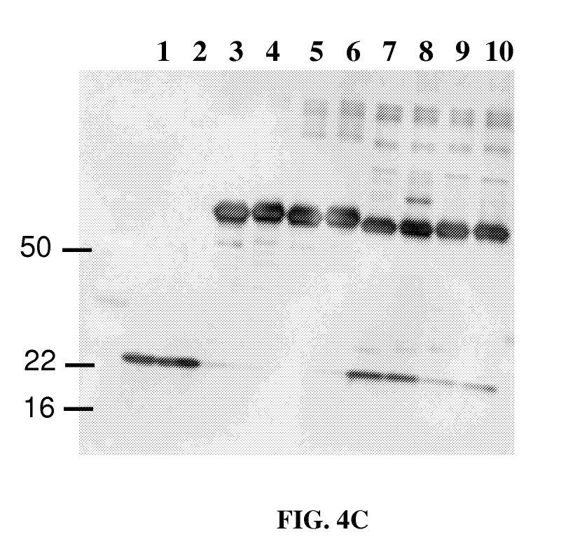

To test the stability of the sFcRn molecule, single chain constructs can be made wherein the heavy chain and light chain subunits are covalently linked by an amino acid linker to form a single chain protein. The single chain constructs can be covalently linked in the heavy chain-linker-light chain orientation or in the light chain-linker-heavy chain orientation (see FIG. 4A).

Heavy Chain-Linker-Light Chain Orientation

The heavy chain-linker-light chain construct is designed to express single chain sFcRn protein in the following orientation: NT-heavy chain-(GGGGS)ₙ linker-light chain-CT (GGGGS is SEQ ID NO: 22) wherein n can vary from one to five copies.

The heavy chain sFcRn open reading frame (ORF) was PCR-amplified using the following primer pairs: Pair A was FCRN-KPNI-BSIWI-F (ATCAGGTACCCGTACGGCCGCCACCATGGGGGTCCCGCGGCCTC (SEQ ID NO:23) and FCRN-2xLINKER-BAMHI-R (AGTCGGATCCGCCTCCGCCGCTGCCTCCTCCGCCGGAGGACTTGGCTGGAGATTCC (SEQ ID NO:24)); and Pair B was FCRN-KPNI-BSIWI-F and FCRN-3xLINKER-BAMHI-R (AGTCGGATCCTCCTCCGCCGCTGCCTCCTCCGCCGCTGCCTCCTCCGCCGGAGGACT TGGCTGGAGATTCC (SEQ ID NO:25)). To prevent restriction cleavage of the heavy chain sFcRn ORF, the heavy chain sFcRn ORF has been previously modified by site-directed mutagenesis to eliminate an internal BamHI restriction site (GGATCC changed to GGCTCC). The PCR-amplified fragments were cloned into the KpnI and BamHI sites of PCDNA™3.1(+) (Invitrogen). The resulting constructs were designated sFcRn/2xlinker/pCDNA3.1 or sFcRn/3xlinker/pCDNA3.1 when using DNA generated with primer Pair A or B, respectively.

The light chain (β2m) sFcRn ORF was PCR-amplified using the following primer pairs: Pair C was B2M-BAMHI-F (AGTCGGATCCATCCAGCGTACTCCAAAGATTCAGG (SEQ ID NO:26)) and B2M-NOTI-MFEI-R (ATCGGCGGCCGCCAATTGTTACATGTCTCGATCCCACTTAACTATCTTGG (SEQ ID NO:27)); and Pair D was B2M-BAMH1-2XLINKER-F (AGTCGGATCCGGCGGAGGAGGCAGCGGCGGAGGCGGCTCCATCCAGCGTACTCCA AAGATTCAGG (SEQ ID NO:28)) and B2M-NOTI-MFEI-R. These primers will generate a BamHI site at the 5′ end and a NotI site at the 3′ end. The PCR-amplified fragments were cloned into the BamHI and NotI sites of sFcRn/2xlinker/pCDNA3.1 or sFcRn/3xlinker/pCDNA3.1. Cloning of DNA generated by primer Pair C into sFcRn/2xlinker/pCDNA3.1 or sFcRn/3xlinker/pCDNA3.1 generated sFcRn/2xlinker/β2M/pcDNA3.1 or sFcRn/3xlinker/β2M/pcDNA3.1, respectively. Cloning of DNA generated by primer Pair D into sFcRn/2xlinker/pCDNA3.1 or sFcRn/3xlinker/pCDNA3.1 generated sFcRn/4xlinker/β2M/pcDNA3.1 or sFcRn/5xlinker/β2M/pcDNA3.1, respectively.

Light Chain-Linker-Heavy Chain Orientation

The light chain-linker-heavy chain construct is designed to express single chain sFcRn protein in the following orientation: NT-light chain-(GGGGS)ₙ linker-heavy chain-CT, wherein n can vary from one to five copies.

To create this construct the β2m ORF was PCR-amplified using the following primer pairs: Pair A was B2M-KPNI-BSIWI-F (CAGGTACCCGTACGGCCGCCACCATGTCTCGCTCCGTGGCCTTAG (SEQ ID NO:29)) and B2M-2xLINKER-BAMHI-R (ATCAGGATCCGCCTCCGCCGCTGCCTCCTCCGCCCATGTCTCGATCCCACTTAACTA TCTTGG (SEQ ID NO:30)); and Pair B was B2M-KPNI-BSIWI-F and B2M-3xLINKER-BAMHI-R (ATCAGGATCCGCCTCCGCCGCTGCCTCCTCCGCCGCTGCCTCCTCCGCCCATGTCTC GATCCCACTTAACTATCTTGG (SEQ ID NO:31)). The PCR-amplified fragments were subcloned into the KpnI and BamHI sites of PCDNA™3.1(+). The resulting constructs were designated β2M/2xlinker/pCDNA3.1 or β2M/3xlinker/pCDNA3.1 when using DNA generated with primer Pair A or B, respectively.

The sFcRn heavy chain ORF was PCR-amplified using the following primer pairs: Pair C was FCRN-BCLI-F (ACGTACTGATCAGCAGAAAGCCACCTCTCCCTCC (SEQ ID NO:32)) and FCRN-NOTI-MFEI-R (ATCGGCGGCCGCCAATTGTTAGGAGGACTTGGCTGGAGATTCC (SEQ ID NO:33)); and Pair D was FCRN-BCL1-2XLINKER-F (ATCAGCTGATCAGGCGGAGGAGGCAGCGGCGGAGGAGGCAGCGCAGAAAGCCACC TCTCCCTCC (SEQ ID NO:34)) and FCRN-NOTI-MFEI-R. These primers will generate a BclI restriction site at the 5′ end and an MfeI site at the 3′ end. Restriction digestion of BclI generates an overhang compatible with BamHI, but upon ligation both sites are lost. A BamHI site (GGATCC) will encode a glycine (GGA) and a serine (TCC), which are part of the amino acid linker repeat. Upon ligation of the BamHI/BclI overhangs, a GGATCA site is formed, which also encodes a glycine (GGA) and a serine (TCA).

The PCR-amplified fragments were cloned into the BamHI and NotI sites of β2M/2xlinker/pCDNA3.1 or β2M/3xlinker/pCDNA3.1. Cloning of DNA generated by primer Pair C into β2M/2xlinker/pCDNA3.1 or β2M/3xlinker/pCDNA3.1 generated β32M/2xlinker/sFcRn/pcDNA3.1 or β2M/3xlinker/sFcRn/pcDNA3.1, respectively. Cloning of DNA generated by primer Pair D into β2M/2xlinker/pCDNA3.1 or β2M/3xlinker/pCDNA3.1 generated β2M/4xlinker/sFcRn/pcDNA3.1 or β2M/5xlinker/sFcRn/pcDNA3.1, respectively.

Expression and Purification of Single Chain sFcRn Proteins

Constructs expressing single chain sFcRn proteins were transfected in HEK 293-H cells (Invitrogen) grown in suspension using a Calcium Phosphate Transfection Kit (Invitrogen). A total of 2.5×10⁷ cells (1×10⁶ cells/ml) were transfected with 25 μg of DNA. Cells were harvested when the cell count reached saturation, usually 6 days. Cell media was cleared by centrifugation and filtration.

Filtered media, typically 50 mL, was concentrated using an AMICON® Ultra-15 device (Millipore) to ˜10 mL. Approximately 10 ml of concentrated media was adjusted to 150 mM MES, pH 6, prior to addition of 400 μl of IgG SEPHAROSE™ 6 Fast Flow resin (GE Healthcare). The media/resin mix was incubated at 4° C. overnight and subsequently washed with 10 volumes of 50 mM MES and 100 mM NaCl at pH 6. sFcRn protein was eluted from the resin using 50 mM phosphate at pH 8.

Samples from the expression and purification of single chain sFcRn proteins were analyzed by SDS-PAGE and Western Blot, as illustrated in FIGS. 4B and 4C, respectively. Lanes 1 and 2 contain wild-type sFcRn protein isolated from CHO cells and HEK 293-H cells, respectively. Lanes 3 through 6 contain the light chain-linker-heavy chain orientation constructs, with 2, 3, 4, and 5 GGGGS (SEQ ID NO: 22) linkers, respectively. Lanes 7 through 10 contain the heavy chain-linker-light chain orientation constructs, with 2, 3, 4, and 5 GGGGS (SEQ ID NO: 22) linkers, respectively. These results illustrate that the single chain sFcRn proteins can be transiently expressed in HEK 293-H cells and purified using IgG SEPHAROSE™ 6 Fast Flow resin.

### Example 4

### Cloning and Expression of Single Chain sFcRn Proteins Carrying a C48A or C251A Heavy Chain Mutation

To determine whether sFcRn can be more favorably attached to the support surface, single chain sFcRn constructs can be made having a single free cysteine residue, by substitution of the cysteine at position 48 or 251 with alanine. These constructs will express either C48A- or C251A-sFcRn, wherein the two subunits are covalently linked by an amino acid linker to form a single chain protein (light chain-linker-heavy chain orientation).

Mutations C48A or C251A were introduced in sFcRn by site-directed mutagenesis using primer pairs FCRN-C48A-F/FCRN-C48A-R or FCRN-C251A-F/FCRN-C251A-R, respectively. The C48A-sFcRn heavy chain ORF was PCR-amplified with primers FCRN-NOTI-MFEI-R/FCRN-BCLI-F and subcloned into the NotI/BamHI sites of β2M/3xlinker/pCDNA3.1 (described above) to generate β2M/3xlinker/C48A-sFcRn/pcDNA3.1. The C251A-sFcRn heavy chain ORF was PCR-amplified with primers FCRN-NOTI-MFEI-R/FCRN-BCLI-F and subcloned into the NotI/BamHI sites of β2M/3xlinker/pCDNA3.1 to generate β2M/3xlinker/C251A-sFcRn/pcDNA3.1.

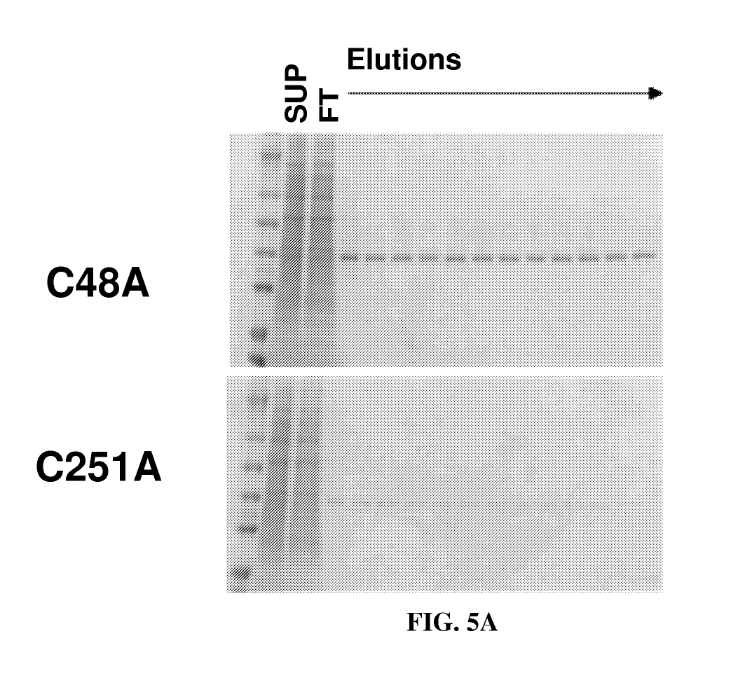

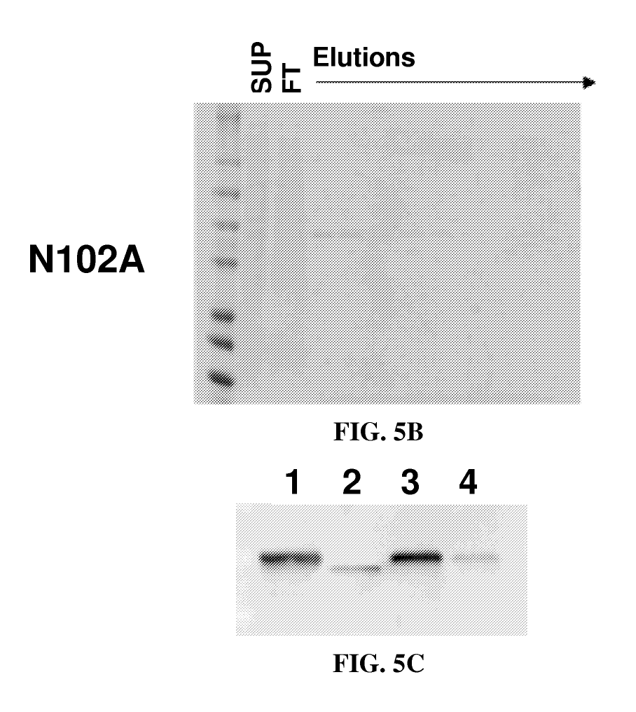

The C48A- and C251A-sFcRn variants were expressed and purified as described above. Samples from the expression and purification of these variants were analyzed by SDS-PAGE and Western Blot, as illustrated in FIGS. 5A and 5C, respectively. Retained fractions from elution of the C48A-sFcRn (250 μg; 0.34 mg/mL) and C251A-sFcRn (51 μg; 0.34 mg/mL) variants were run on an SDS-PAGE gel. The transiently expressed C48A- and C251A-sFcRn variants eluted as single bands on SDS-PAGE gels (see FIG. 5A, lanes marked “Elutions”). Lanes 1 and 2 contain samples of the supernatant and flow-through from the purification procedure (see FIG. 5A, lanes marked “SUP” and “FT”). The purified C48A- and C251A-sFcRn variants were subsequently visualized by immunoblotting with an anti-sFcRn antibody (see FIG. 5C, Lanes 3 and 4, respectively). Lane 1 contains a sample of single chain sFcRn. These results illustrate that the C48A- and C251A-sFcRn variants can be transiently expressed in HEK 293-H cells and purified using IgG SEPHAROSE™ 6 Fast Flow resin.

### Example 5

### Cloning and Expression of Single Chain sFcRn Proteins Carrying a N102A Heavy Chain Mutation

To determine whether reduced sFcRn heterogeneity can affect sFcRn stability, single chain sFcRn glycosylation variants can be made by substitution of the asparagine at position 102 with alanine. This construct will express N102A-sFcRn, wherein the two subunits are covalently linked by an amino acid linker to form a single chain protein (light chain-linker-heavy chain orientation), and will not be glycosylated at Asn^{t02} following expression in HEK 293-H cells.

The N102A-sFcRn variant was expressed and purified as described above. Samples from the expression and purification of these variants were analyzed by SDS-PAGE and Western Blot, as illustrated in FIGS. 5B and 5C, respectively. Retained fractions from elution of the N102A-sFcRn (6.65 μg; 0.19 mg/mL) variant were run on an SDS-PAGE gel. The transiently expressed N102A-sFcRn variant eluted as a single band on an SDS-PAGE gel (see FIG. 5B, lanes marked “Elutions”). Lanes 1 and 2 contain samples of the supernatant and flow-through from the purification procedure (see FIG. 5B, lanes marked “SUP” and “FT”). The purified N102A-sFcRn variant was subsequently visualized by immunoblotting with an anti-sFcRn antibody (see FIG. 5C, Lane 2). Lane 1 contains a sample of single chain sFcRn. These results illustrate that the N102A-sFcRn variant can be transiently expressed in HEK 293-H cells and purified using IgG SEPHAROSE™ 6 Fast Flow resin; however, the low level of expression may indicate that this glycosylation residue is important for expression of the protein.

### Example 6

### Cloning and Expression of Single Chain sFcRn Heterodimer Dimer Proteins

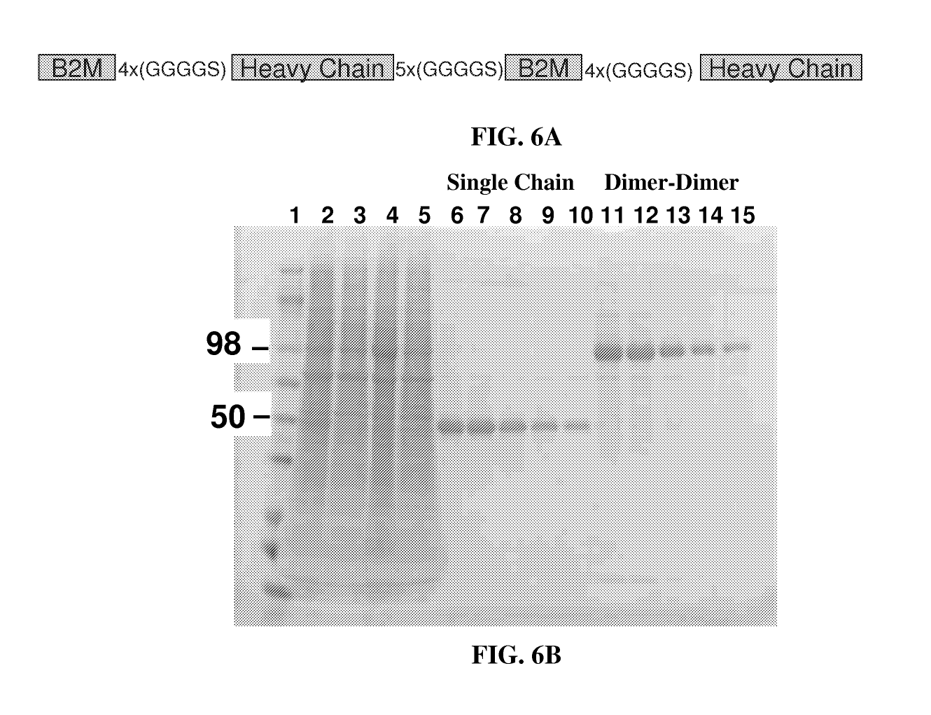

To determine whether the affinity of the sFcRn molecule for Fc-containing proteins can be increased, sFcRn heterodimer dimer constructs can be made wherein two heavy chain and light chain subunits are covalently linked by an amino acid linker to form a dimerized single chain protein. The sFcRn heterodimer dimer constructs can be covalently linked in the light chain-linker-heavy chain-linker-light chain-linker-heavy chain orientation (see FIG. 6A).

To create this construct a DNA fragment was generated by PCR-amplification of pcDNA3.1/sFcRn-5xlinker-β2M using primers FCRN-BCL1-F2 (ATGGAATGATCATTCCACGCCTCGTCGTCAC (SEQ ID NO:35)) and B2M-XBAI-R (AGCATCTAGAGTAAACCTGAATCTTTGGAGTACGCTG (SEQ ID NO:36)). FCRN-BCL1-F2 will anneal to the heavy chain sFcRn and generate a BclI overhang. B2M-XBAI-R will anneal to β2m and introduce an XbaI overhang. The PCR-amplified fragment was subcloned into the BamH1 and XbaI sites of pcDNA3.1/β2M-4-xlinker-sFcRn, generating an intermediate construct. Next, another DNA fragment was PCR-amplified using pcDNA3.1/β2M-4-xlinker-sFcRn as template and primers B2M-XBA1-F (TCGTTCTAGACATCCAGCAGAGAATGGAAAGTC (SEQ ID NO:37)), which anneals to FcRn, and FCRN-XBA1-R(CCCTCTAGACTCGAGCGGCC (SEQ ID NO:38)), which anneals downstream of the sFcRn stop codon. This DNA fragment was subcloned into the XbaI site of the intermediate construct described above, generating pcDNA3.1/β2M-4-xlinker-sFcRn-5xlinker-β2M-4-xlinker-sFcRn.

The sFcRn heterodimer dimer construct was expressed and purified as described above. Samples from the expression and purification of this construct and single chain sFcRn were analyzed by SDS-PAGE, as illustrated in FIG. 6B. Retained fractions from elution of the sFcRn heterodimer dimer construct (690 μg; 0.33 mg/mL) and single chain sFcRn light chain-linker-heavy chain construct (310 μg; 0.31 mg/mL) were run on an SDS-PAGE gel. The transiently expressed sFcRn heterodimer dimer construct (Lanes 11-15) and single chain sFcRn (Lanes 6-10) eluted as single bands on an SDS-PAGE gel. Lanes 2 through 5 contain samples of the supernatant and flow-through of the single chain sFcRn (Lanes 2 and 3) and sFcRn heterodimer dimer (Lanes 4 and 5) constructs from the purification procedure. Lane 1 contains molecular weight markers (SEEBLUE® Plus 2). These results illustrate that the sFcRn heterodimer dimer construct can be transiently expressed in HEK 293-H cells and purified using IgG SEPHAROSE™ 6 Fast Flow resin.

### Example 7

### Cloning and Expression of Disulfide Bond-Linked Single Chain sFcRn Proteins

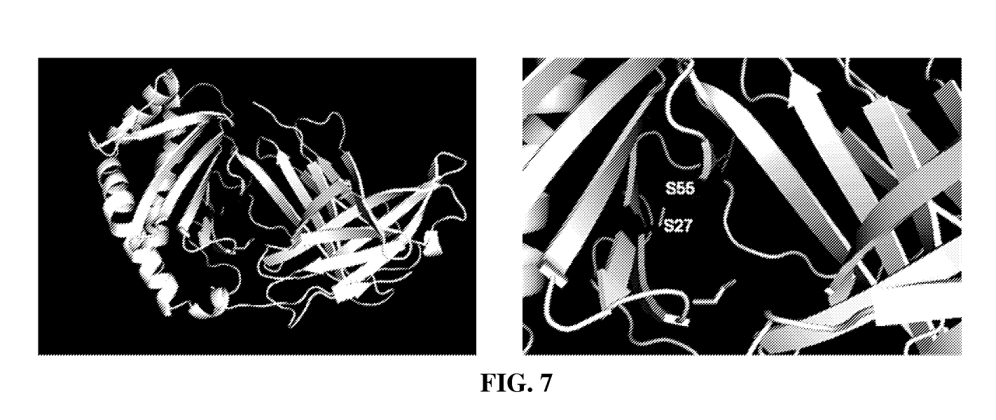

To test the stability of the sFcRn molecule, disulfide bond-linked single chain sFcRn constructs can be made wherein the heavy chain and light chain subunits are covalently linked by a recombinantly engineered disulfide bond to form a single chain protein. The disulfide bond-linked single chain sFcRn constructs can be made by substitution of, for example, the serines at positions 27 or 55 with cysteines (see FIG. 7). This construct will express S27C-S55C-sFcRn, wherein the two subunits are covalently linked by a disulfide bond to form a single chain protein.

Embodiments of the invention (E) include E1-E29:

E1. A method of purifying an Fc-containing protein from a sample, the method comprising:
(a) contacting a sample containing said Fc-containing protein with soluble neonatal Fc receptor (sFcRn) bound to a support surface under conditions that allow said Fc-containing protein to bind sFcRn;
(b) separating said Fc-containing protein from the sample;
(c) dissociating said Fc-containing protein from the sFcRn; and
(d) collecting said Fc-containing protein.

E2. A method of purifying an enzymatically-active protein:Fc fusion protein from a sample, the method comprising:
(a) contacting a sample containing said fusion protein with soluble neonatal Fc receptor (sFcRn) bound to a support surface under conditions that allow said fusion protein to bind sFcRn;
(b) separating said fusion protein from the sample;
(c) dissociating said fusion protein from the sFcRn; and
(d) collecting said fusion protein.

E3. A method of purifying a Factor VIII:Fc fusion protein from a sample, the method comprising:
(a) contacting a sample containing said fusion protein with soluble neonatal Fc receptor (sFcRn) bound to a support surface under conditions that allow said fusion protein to bind sFcRn;
(b) separating said fusion protein from the sample;
(c) dissociating said fusion protein from the sFcRn; and
(d) collecting said fusion protein.

E4. A method of increasing the biological activity of an Fc fusion protein in a sample, the method comprising:
(a) contacting a sample containing said fusion protein with soluble neonatal Fc receptor (sFcRn) bound to a support surface under conditions that allow said Fc fusion protein to bind sFcRn;
(b) separating said Fc fusion protein from the sample;
(c) dissociating said Fc fusion protein from the sFcRn; and
(d) collecting said Fc fusion protein.

E5. A method according to E4, wherein said Fc fusion protein is selected from the group consisting of an enzymatically active protein:Fc fusion protein and a Factor VIII:Fc fusion protein.

E6. The method of any one of E1 to E5, wherein the percent purity, by weight, of said Fc-containing protein collected, compared to the sum weight of associated contaminants, is a percent purity selected from the group consisting of:
(a) at least about 20% pure;
(b) at least about 30% pure;
(c) at least about 40% pure;
(d) at least about 50% pure;
(e) at least about 60% pure;
(f) at least about 70% pure;
(g) at least about 80% pure;
(h) at least about 85% pure;
(i) at least about 90% pure;
(j) at least about 95% pure,
(k) at least about 98% pure; and
(l) at least about 99% pure.

E7. The method of any one of E1 to E6, wherein the percent recovery, by weight, of said Fc-containing protein collected, compared to the sum weight of Fc-containing protein in said sample, is a percent recovery selected from the group consisting of:
(a) at least about 20% recovery;
(b) at least about 30% recovery;
(c) at least about 40% recovery;
(d) at least about 50% recovery;
(e) at least about 60% recovery;
(f) at least about 70% recovery;
(g) at least about 80% recovery;
(h) at least about 85% recovery;
(i) at least about 90% recovery;
(j) at least about 95% recovery,
(k) at least about 98% recovery; and
(l) at least about 99% recovery.

E8. The method of any one of E1 to E7, wherein the fold increase in the biological activity of said Fc-containing protein collected, compared to the biological activity of Fc-containing protein in said sample, is a fold increase selected from the group consisting of:
(a) at least about 2-fold;
(b) at least about 5-fold;
(c) at least about 10-fold;
(d) at least about 20-fold;
(e) at least about 50-fold;
(f) at least about 100-fold;
(g) at least about 200-fold;
(h) at least about 500-fold;
(i) at least about 1000-fold;
(j) at least about 2000-fold;
(k) at least about 5000-fold; and
(l) at least about 10000-fold.

E9. The method of any one of E1 to E8, wherein one or both heavy chain (α-chain) or light chain (β2m) domains of said sFcRn are modified to increase or decrease the affinity of Fc-containing protein binding.

E10. The method of E9, wherein said modification comprises one or more amino acid substitutions, deletions, or insertions.

E11. The method of E9, wherein said modification comprises a post-translational chemical modification.

E12. The method of any one of E1 to E11, wherein the modification increases the binding affinity of sFcRn to Fc-containing proteins.

E13. The method of any one of E1 to E12, wherein one or both α-chain or β2m are modified to modulate linkage to the support surface wherein said modification increases efficiency of Fc-containing protein purification.

E14. The method of any one of E1 to E13, wherein one or both α-chain or β2m are modified to modulate linkage to the support surface wherein said modification increases efficiency of Factor VIII:Fc fusion protein purification.

E15. A method according to E13 or E14 wherein the modification increases sFcRn linkage to the surface.

E16. The method of any one of E1 to E15, wherein one or both α-chain or β2m are modified to increase the stability of sFcRn binding activity.

E17. The method of any one of E1 to E16, wherein the sFcRn α-chain and β2m are chemically, photo, or thermally cross-linked.

E18. The method of any one of E1 to E17, wherein the sFcRn α-chain and β2m are covalently linked by an amino acid linker.

E19. A method according to E18, wherein the amino acid linker comprises (GGGGS)n, wherein n is between 1 and 5.

E20. The method of any one of E1 to E19, wherein the sFcRn is human sFcRn.

E21. The method of any one of E1 to E20, wherein the support surface comprises SEPHAROSE™, agarose, silica, collodion charcoal, sand, polystyrene, or methacrylate.

E22. The method of any one of E1 to E21, wherein sFcRn is bound to a support surface through formation of a covalent bond.

E23. A method according to E22 wherein the bond formation comprises an amide bond, an amine bond, a carbon bond, a disulfide bond, an ester bond, an ether bond, a thioether bond, a urea bond, or a thiourea bond.

E24. The method of any one of E1 to E23, wherein sFcRn is bound to a support surface through a chemically, photo, or thermally activated cross-link.

E25. The method of any one of E1 to E24, wherein said method comprises contacting the sample containing said Fc-containing protein with sFcRn at or below a pH of about 7.0.

E26. A method according to E25, wherein said method comprises contacting the sample containing said Fc-containing protein with sFcRn at or below a pH of about 6.9, 6.8, 6.7, 6.6, 6.5, 6.4, 6.3, 6.2, 6.1, 6.0, 5.9, 5.8, 5.7, 5.6, 5.5, 5.0, 4.5, or 4.0.

E27. The method of any one of E1 to E26, wherein said method comprises dissociating the Fc-containing protein from sFcRn at or above a pH of about 7.0.

E28. A method according to E27, wherein said method comprises dissociating the Fc-containing protein from sFcRn at or above a pH of about 7.1, 7.2, 7.3, 7.4, 7.5, 7.6, 7.7, 7.8, 7.9, 8.0, 8.1, 8.2, 8.3, 8.4, 8.5, 9.0, 9.5, or 10.0.

E29. The method of any one of E1 to E28, wherein said method comprises one or more washing steps prior to dissociating the Fc-containing protein from the sFcRn.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method of purifying an Fc-containing protein from a sample, the method comprising:
(a) contacting a sample containing said Fc-containing protein with soluble neonatal Fc receptor (sFcRn) bound to a support surface under conditions that allow said Fc-containing protein to bind sFcRn;
(b) separating said Fc-containing protein from the sample;
(c) dissociating said Fc-containing protein from the sFcRn; and
(d) collecting said Fc-containing protein.

2. The method of claim 1, wherein said Fc-containing protein is an enzymatically-active protein:Fc fusion protein.

3. The method of claim 1, wherein said Fc-containing protein is a Factor VIII:Fc fusion protein.

4. A method of increasing the biological activity of an Fc fusion protein in a sample, the method comprising:
(a) contacting a sample containing said fusion protein with soluble neonatal Fc receptor (sFcRn) bound to a support surface under conditions that allow said Fc fusion protein to bind sFcRn;
(b) separating said Fc fusion protein from the sample;
(c) dissociating said Fc fusion protein from the sFcRn; and
(d) collecting said Fc fusion protein.

5. The method according to claim 4, wherein said Fc fusion protein is selected from the group consisting of an enzymatically active protein:Fc fusion protein and a Factor VIII:Fc fusion protein.

6. The method of claim 1, wherein the percent purity, by weight, of said Fc-containing protein collected, compared to the sum weight of associated contaminants, is a percent purity selected from the group consisting of:
(a) at least about 20% pure;
(b) at least about 30% pure;
(c) at least about 40% pure;
(d) at least about 50% pure;
(e) at least about 60% pure;
(f) at least about 70% pure;
(g) at least about 80% pure;
(h) at least about 85% pure;
(i) at least about 90% pure;
(j) at least about 95% pure,
(k) at least about 98% pure; and
(l) at least about 99% pure.

7. The method of claim 1, wherein the percent recovery, by weight, of said Fc-containing protein collected, compared to the sum weight of Fc-containing protein in said sample, is a percent recovery selected from the group consisting of:
(a) at least about 20% recovery;
(b) at least about 30% recovery;
(c) at least about 40% recovery;
(d) at least about 50% recovery;
(e) at least about 60% recovery;
(f) at least about 70% recovery;
(g) at least about 80% recovery;
(h) at least about 85% recovery;
(i) at least about 90% recovery;
(j) at least about 95% recovery,
(k) at least about 98% recovery; and
(l) at least about 99% recovery.

8. The method of claim 4, wherein the fold increase in the biological activity of said Fc fusion protein collected, compared to the biological activity of Fc fusion protein in said sample, is a fold increase selected from the group consisting of:
(a) at least about 2-fold;
(b) at least about 5-fold;
(c) at least about 10-fold;
(d) at least about 20-fold;
(e) at least about 50-fold;
(f) at least about 100-fold;
(g) at least about 200-fold;
(h) at least about 500-fold;
(i) at least about 1000-fold;
(j) at least about 2000-fold;
(k) at least about 5000-fold; and
(l) at least about 10000-fold.

9. The method of claim 1, wherein one or both heavy chain (α-chain) or light chain (β2m) domains of said sFcRn are modified to increase or decrease the affinity of Fc-containing protein binding.

10. The method of claim 9, wherein said modification comprises a change selected from the group consisting of:
(a) one or more amino acid substitutions;
(b) one or more amino acid deletions;
(c) one or more amino acid insertions;
(d) a post-translational chemical modification; and
(e) a modification which increases the binding affinity of sFcRn to Fc-containing proteins.

11. (canceled)

12. (canceled)

13. The method of claim 1, wherein one or both heavy chain (α-chain) or light chain (β2m) domains of said FcRn are modified to modulate linkage to the support surface wherein said modification increases efficiency of Fc-containing protein purification.

14. (canceled)

15. The method according to claim 13 wherein the modification increases sFcRn linkage to the surface.

16. (canceled)

17. The method of claim 1, wherein one or both heavy chain (α-chain) or light chain (β2m) domains of said FcRn are modified to increase the stability of sFcRn binding activity.

18. The method of Claim 1, wherein the sFcRn α-chain and β2m are chemically, photo, or thermally cross-linked.

19. The method of claim 1, wherein the sFcRn α-chain and β2m are covalently linked by an amino acid linker.

20. The method according to claim 19, wherein the amino acid linker comprises (GGGGS)n, wherein n is between 1 and 5.

21. The method of claim 1, wherein the sFcRn is a human sFcRn.

22. The method of claim 1, wherein the support surface comprises SEPHAROSE™, agarose, silica, collodion charcoal, sand, polystyrene, or methacrylate.

23. The method of claim 1, wherein the sFcRn is bound to a support surface through formation of a bond selected from the group consisting of:
(a) a covalent bond;
(b) an amide bond;
(c) an amine bond;
(d) a carbon bond;
(e) a disulfide bond;
(f) an ester bond;
(g) an ether bond;
(h) a thioether bond;
(i) a urea bond; and
(j) a thiourea bond.

24. (canceled)

25. The method of claim 1, wherein the sFcRn is bound to a support surface through a chemically, photo, or thermally activated cross-link.

26. The method of claim 1, wherein said method comprises contacting the sample containing said Fc-containing protein with sFcRn at or below a pH selected from the group consisting of:
(a) about 7.0;
(b) about 6.9;
(c) about 6.8;
(d) about 6.7;
(e) about 6.6;
(f) about 6.5;
(g) about 6.4;
(h) about 6.3;
(i) about 6.2;
(j) about 6.1;
(k) about 6.0;
(l) about 5.9;
(m) about 5.8;
(n) about 5.7;
(o) about 5.6;
(p) about 5.5;
(q) about 5.0;
(r) about 4.5; and
(s) about 4.0.

27. (canceled)

28. The method of claim 1, wherein said method comprises dissociating the Fc-containing protein from sFcRn at or above a pH selected from the group consisting of:
(a) about 7.0;
(b) about 7.1;
(c) about 7.2;
(d) about 7.3;
(e) about 7.4;
(f) about 7.5;
(g) about 7.6;
(h) about 7.7;
(i) about 7.8;
(j) about 7.9;
(k) about 8.0;
(l) about 8.1;
(m) about 8.2;
(n) about 8.3;
(o) about 8.4;
(p) about 8.5;
(q) about 9.0;
(r) about 9.5; and
(s) about 10.0.

29. (canceled)

30. The method of claim 1, wherein said method comprises one or more washing steps prior to dissociating the Fc-containing protein from the sFcRn.
